(19) United States
(12) Patent Application Publication
(10) Pub. No.: US 2015/0164377 A1
(43) Pub. Date: Jun. 18, 2015

(54) SYSTEM AND METHOD OF BODY MOTION ANALYTICS RECOGNITION AND ALERTING

(71) Applicants: Vaidhi Nathan, San Jose, CA (US); Anoo Nathan, San Jose, CA (US)

(72) Inventors: Vaidhi Nathan, San Jose, CA (US); Anoo Nathan, San Jose, CA (US)

(21) Appl. No.: 14/207,495

(22) Filed: Mar. 12, 2014

Related U.S. Application Data
(60) Provisional application No. 61/801,737, filed on Mar. 15, 2013.
(60) Provisional application No. 61/779,608, filed on Mar. 13, 2013.

(51) Int. Cl. A61B 5/11; A61B 5/00

(52) U.S. Cl. CPC A61B 5/1122; A61B 5/6802; A61B 5/4094

(57) ABSTRACT

Device, system and methods for using body worn sensors to analyze human body motion. The device, often configured to be worn on the user's wrist, arm, neck, belt or other location, comprises a processor, an output device (often a wireless transceiver), and at least one accelerometer, angle, location, direction, or physiological state sensor. The processor may be configured for various functions, such as analyzing habitual user activities, establishing normal baselines, classifying types of motion and reporting, and logging sensor readings or analysis results. Various algorithms may be used to determine when significant deviations from baseline values occur, and, depending on the type of deviation, the output device can transmit data and alerts. In some embodiments, the data and alerts may be further analyzed by other computerized devices such as mobile phones (smartphones), computers, internet servers, and the like.

### CROSS REFERENCE TO RELATED APPLICATIONS

This application claims the priority benefit of US provisional applications 61/779,608, inventor Vaidhi Nathan, file Mar. 13, 2013, and 61/801,737, inventor Vaidhi Nathan, filed Mar. 15, 2013, the complete contents of both applications are incorporated herein by reference.

### BACKGROUND OF THE INVENTION

1. Field of the Invention

This invention is in the field of personal monitoring devices, systems, and methods.

2. Description of the Related Art

The background of this invention is related to human body analysis. Although various types of human worn sensors for purposes of health monitoring, fitness, and other purposes are known, typically prior art devices used only a small number of sensors. Some of these various prior art inventions or solutions also did data collection from the sensors. Some also provided various types of messages, again often based on single sensors and threshold levels.

The inventor's previous work in this field includes the disclosures of Nathan et. al., U.S. patent application Ser. No. 12/154,085, now U.S. Pat. No. 8,075,499, the complete contents of which are incorporated herein by reference. See U.S. application Ser. No. 12/154,085 for a more extensive background discussion.

### BRIEF SUMMARY OF THE INVENTION

The present invention typically uses multiple sensors, more data, and more advanced analytics than prior art in this field. The present invention can thus often provide more advanced classifying details, and also produce more meaningful results and/or summaries.

This invention also integrates both physical sensors and biological sensors to join into new level of analysis and algorithms explained here. This invention is a notable step forward in creating more intelligent and complex analysis of multi-sensor application to analyze human body part motion.

More specifically, the present invention is related to human body motion analysis (which can include movements of hands, legs, head, torso) using body worn sensors. This analysis is focused on understanding movements, detecting and classifying the type of motion and sending messages, reports and alerts. The present disclosure describes the invention's methodology, technology and process. In other embodiments, the invention may also be viewed as being a system that assembles various components and provides a functional apparatus for use.

In some embodiments, the invention will use various body physical/geometric sensors, such as one or more sensors such as acceleration sensors (accelerometers), angle sensors (gyros), location sensor (GPS), directional sensor (compass or magnetometers). Additionally, physiological sensors (such as body temperature, heat rate/pulse etc.) can also be used.

The invention will typically employ one or more processors (e.g. compute processors such as microprocessors, and the like) and various types of motion analytics algorithms to analyze the characteristics of various daily normal activities. These normal activities can include walking, running, talking, sleeping and the like. The invention can use data from these various sensors to establish a baseline of what is “normal” for these types of activities. These baselines may be established across groups of individuals and/or customized for each individual. These baselines may also be adjusted for different situations.

In other embodiments, the invention may classify the type of motion and report, and record (log) these results. Processor implemented rules or logic based software can analyze the data, and determines if there is a significant deviation from the baseline or normal values. If such a deviation is detected, the system can then send alerts and/or notifications reporting on these deviations.

The data or alerts or other analytics may be sent from the sensor via a wired or wireless medium to another device or data collection system, such as a mobile phone or hub or computer or network device. Such analytics and determination of abnormal conditions indicating deviations from norm can be done in the body worn sensor, or can be done outside on a connected device such as computer or mobile phone or remotely on the internet/cloud.

### BRIEF DESCRIPTION OF THE DRAWINGS

FIG. 1 shows an example of the invention configured as various types of body worn devices

FIG. 2 shows an example of various types of sensors that may be used in the invention.

FIG. 3 shows an example of some of the more standard hardware components of the invention.

FIG. 4 shows an example of the invention, along with some optional components.

FIG. 5 shows an example of how the invention systems can connect

FIG. 6 shows an example of the overall system.

FIG. 7 shows an example flow chart showing the data flow according to the invention.

FIG. 8A shows an example of 1D Haar wavelets.

FIG. 8B shows some algorithms that may be used in some embodiments of the invention.

FIG. 8C shows some algorithms that may be used in some embodiments of the invention.

FIG. 8D shows some algorithms that may be used in some embodiments of the invention.

FIG. 8E shows some algorithms that may be used in some embodiments of the invention.

FIG. 9 shows a drawing of a type of multi-class classification.

FIG. 10 shows an example of TFD analysis signals.

FIG. 11 shows a TF representation of the signal.

FIG. 12 shows various stages of a fall detection algorithm.

FIG. 13 shows an example of a device only architecture.

FIG. 14 gives an example of a device+mobile phone architecture.

FIG. 15 gives an example of a device+mobile+cloud/internet server architecture.

FIG. 16 gives an example of a cloud (or any external system)+device architecture.

### DETAILED DESCRIPTION OF THE INVENTION

As previously discussed, the background of this invention is related to human body analysis. The prior art only addressed typically a single or 2 sensors. Most past inventions or solutions typically did data collection from these sensors. Some may even give a message based on a single sensor and threshold level. But this invention is more related to using multiple sensors and doing advanced analytics using more data and classifying details and arriving at more meaningful results or summary. This invention is also integrates both physical sensors and biological sensors to join into new level of analysis and algorithms explained here. This invention is a notable step forward in creating more intelligent and complex analysis of multi-sensor application to analyze human body part motion.

In some embodiments, the invention may be a human body worn device or system with one more sensors and a CPU unit. More specifically, the device may be a body worn sensor. For example, the device may be:

1. Worn on wrist as a watch, or bracelet or band
2. Worn as a necklace or pendant or on a neck or torso hanging device
3. on the arm, as an arm band, or
4. attached to a belt
5. attached to the legs or feet
6. placed on the hat or inside cap or helmet on the head
7. placed on the eye glasses as part of the eye glass or frame,
8. attached to the chest or torso by a strap or equivalent or
9. or sown on to the clothes (upper or lower body clothing) or embedded in the fabric
10. on another part of the body

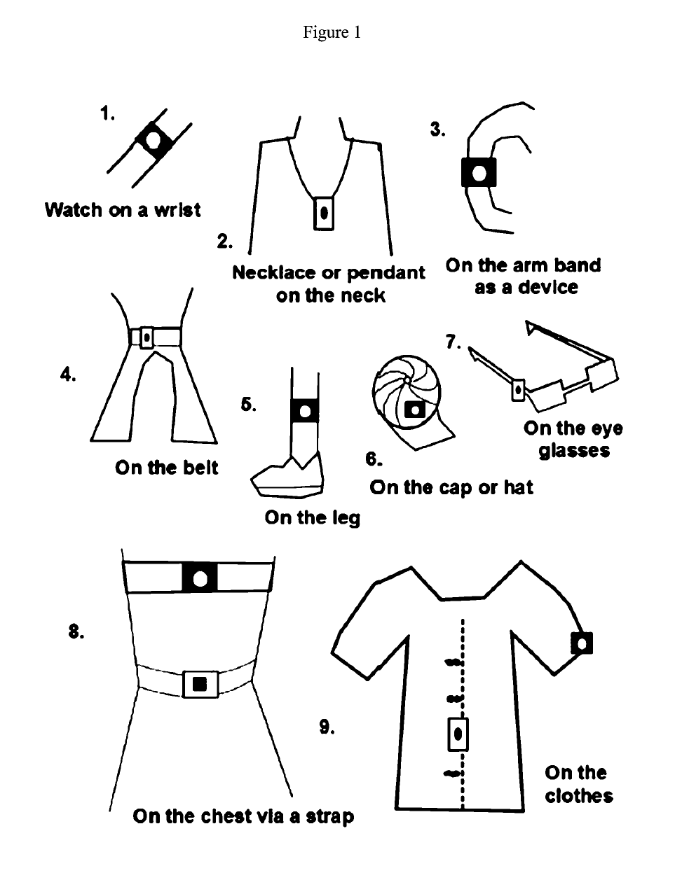

Examples of these various embodiments are shown in FIG. 1.

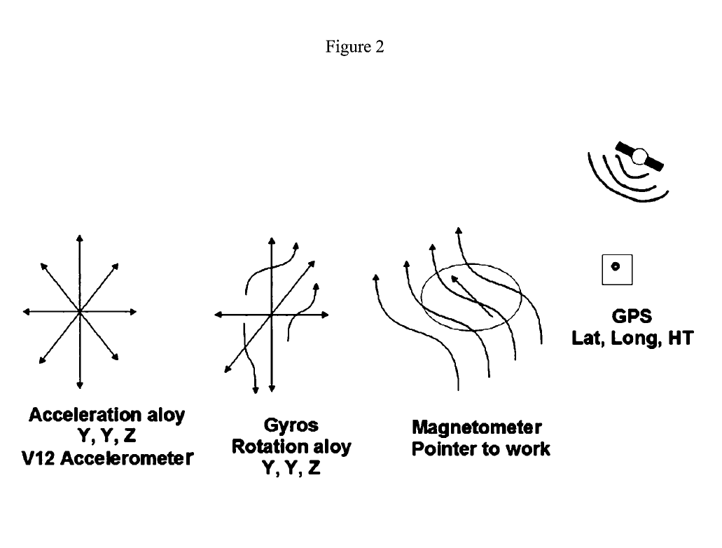

The invention may also include one or more various physical/geometric sensors. These physical sensors may be related to geometric, location, orientation, speed, direction, body acceleration etc., and some of these are shown in FIG. 2. These sensors may include:

3.1: Acceleration sensor (accelerometers). This sensor type measures the acceleration of an object or in the case body part. The sensor will be tied to the body part and that part's acceleration (or rate of change of velocity) will be measured. The accelerometer can be either 2 dimensional (like X & Y axis only) or 3 Dimensional (like X, Y, Z axes).
3.2: Angle sensors, also referred as gyros or gyroscopes or gyro sensors. These sensors measure the angle or tilt of an object, or in this the angle of the body part. The gyro can be either 2 dimensional (I and J, angle of rotation of X or Y axis), or 3 Dimensional like (I, J, K the angle of rotation of X, Y, Z axis respectively).

These acceleration and angle sensors can provide unique geometrical information of the body part. Either one of both can be used in this invention. Additionally, other sensors may include

3.3: Direction or orientation sensors, such as magnetometers. These sensors can measure the direction or orientation to the true north or earth, by detecting the magnetic fields. These sensor materials will align with the earth magnetic fields and hence can help point to north, south, east and west directions. Sometimes this direction information can be useful in our computation.
3.4: GPS (or Global Positioning System). This sensor though is common, but it has been in the past prior art, mostly used as an individual sensor. But in our case, we are using it integrated with other physical and geometric sensors to extract more context specific information. The GPS sensor provides information on the person's location—such as latitude, longitude and altitude/height. With these pieces of information, and by using algorithmic techniques, location of the person can be determined within 10 feet accuracy.

By using combinations of these one or more geometric/physical sensors, more human motions can be analyzed. The final working system can have one or more sensors. The sensor data is combined to facilitate mathematical analysis and advanced algorithms as mentioned in this invention.

In some embodiments, the whole invention can be applied to all 3 forms of applications or utilities or solutions: These may include:
1. A whole system. An independent and self-functioning system (including hardware, firmware, sensors, software and algorithms)
2. A method or methodology, for making technical product and making a viable solution or component
3. Algorithms—primarily the software, math processing and getting detection, recognition and alerting to work and the method to do the software
4. A module with one of more of these method or algorithm or a system component. (including any/some hardware, firmware, sensors, software and algorithms)

Embodiments of these above forms are viable and possible and presented in this invention. A utility patent can be followed or filed based on any of the above embodiments or forms.

In many embodiments, the invention's system hardware may be implemented by an electronic device that is small and includes one or more of these sensors. The system will generally also include a CPU or microprocessor or microcontroller or a central-processing-unit to collect these sensor data, process, store it and send communications out.

Typically the invention's hardware may incorporate four sub-units, such as: the preferred, standard or even mandatory units such as:

4.1: Sensor units: one or more of these sensors
4.2 Computing units: CPU, memory both permanent and random/temporary usage
4.3 Communication unit: This can be one or more of these Wired or wireless communication unit such as Ethernet, Wi-Fi, Bluetooth, Zigbee or 3G/cellular modem units.

All electronics needs a power source such as battery, solar cell, or external power input.

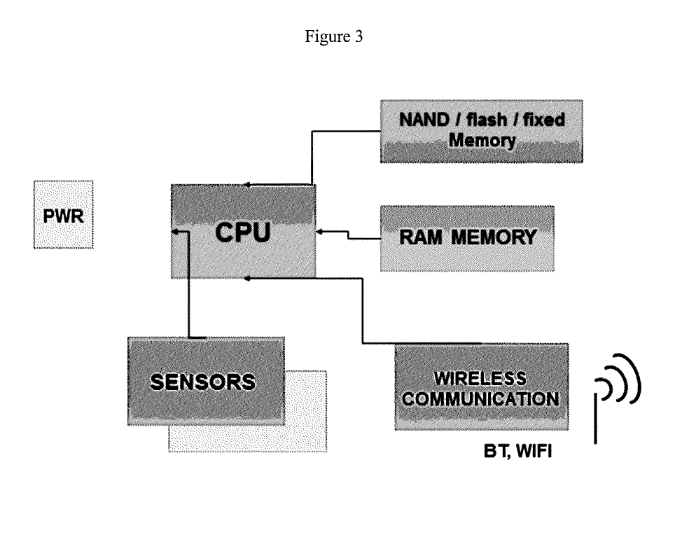

FIG. 3 shows an example of some of the standard hardware components of the invention.

The invention may also comprise other optional units as well. These may include

4.4 Display unit: A Display screen or small visible unit to display a message or monitor or screen. This can be text based or raster image display based. This can be touch screen or display only screen.
4.5 User interaction or I/O. This can be buttons or touch screen or some ways to make user interact with the system or device.
4.6: Audio and vibrator. The vibrator unit buzzes and or vibrates to indicate the user of some message or condition or notification. The Audio has 2 sub components, buzzer or mini speaker and a microphone to listen the user sound or speech. The microphone (mic.) and speaker—goes hand in hand for audio interactions.

Note that these components 4.4, 4.5, 4.6—are optional and the device may or may not have these.

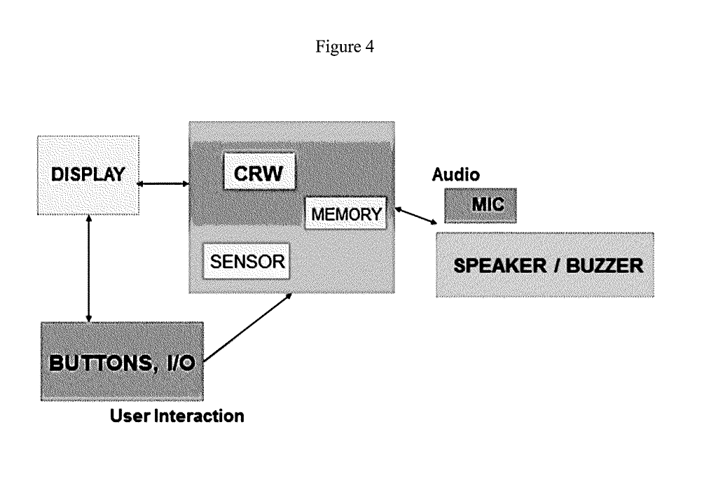

FIG. 4 shows an example of the invention, along with some optional components.

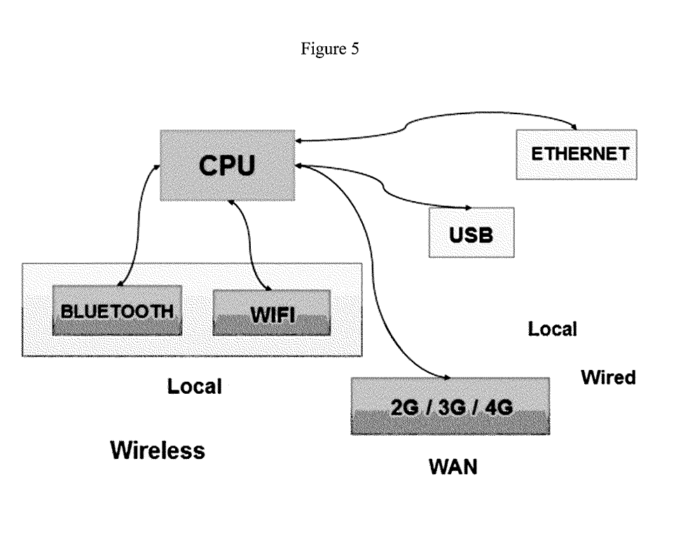

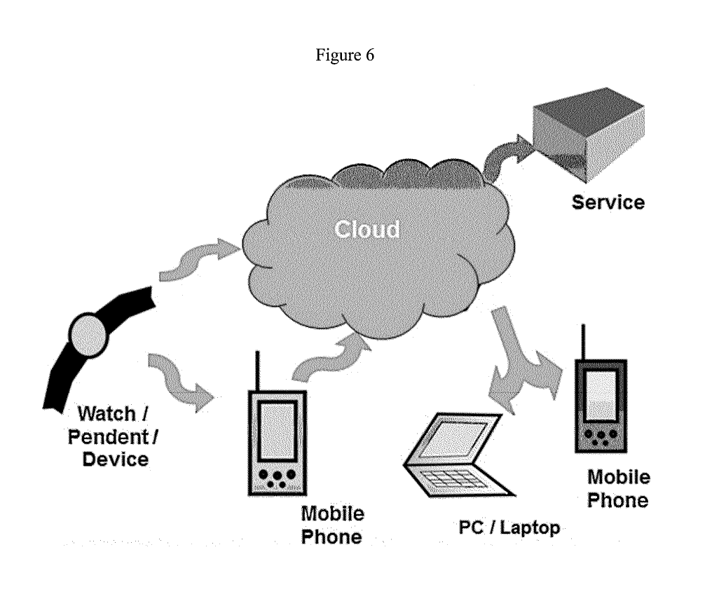

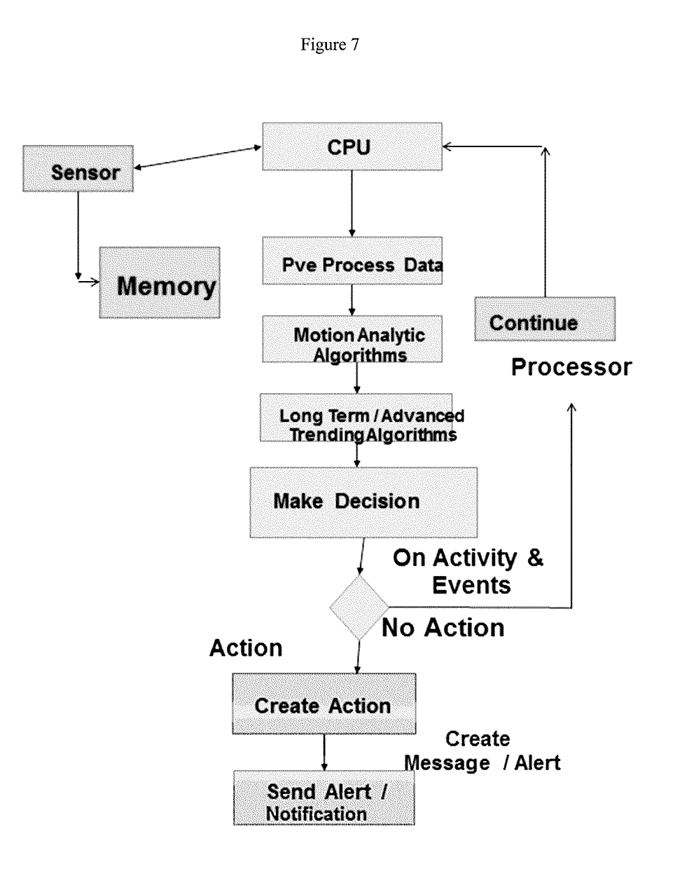

FIG. 5 shows an example of how the various invention systems can connect. FIG. 6 shows an example of the overall system, and FIG. 7 shows an example flow chart showing the data flow according to the invention. These will be discussed in more detail shortly.

### Data Acquisition

Typically the invention will acquire data from one or more of the above sensors. Each sensor may have its own electronic frequency of operations or clock rate. For each example, the acceleration measuring system may operate at 25 or 50 or 100 Hz or times/sec. Other sensors may have the same or different data frequency. The central CPU will poll and collect data from the sensors, each in its own frequency. The data is collected in the raw sensor output form. It can be analog or digital. The CPU will store the data in the local memory or RAM memory temporarily. For each sensor output, the CPU polls, collects data and stores in the memory. Each sensor is stored separately. This data will be used next for pre and post processing, analysis and output.

Data pre-processing, processing and organization. The data from the sensor may go through a preprocessing step. A pre-processing step can be preparing the sensor data to be more viable, useful and meaningful. Many times, the sensor outputs a raw internal physical electronic output value. Such as value may or may not be directly useful. Hence the data is converted or transformed into a more useful value. Such as a sensor may output the speed or parameter between 0 to 1 or 1-100 or 4-20 mA. This data will be converted to a more useful physical measurement like speed, acceleration or position etc.

This is part of the pre-processing, sensor data conversion or transformation. Finally data is organized into the memory, in terms of each sensor, values, time, frequency and ranges etc. Such useful data and well-organized data, is then used in the next step for analysis or mathematical algorithms to understand the body motion.

### Algorithms and Methods to Perform Human Motion Analytics

In the following sections we will disclose methods, mathematical algorithms and various computational techniques to determine and process human body motion analytics. This section is divided into various sub sections as follows:
i. Algorithm types: Rules, Formulae and analytics method vs. Learning and classification methods
ii. Short and long term analytics: Short term determines the body motion over a smaller period (a small hand movement) vs. long term determines the human action or activity over a longer time (like sleeping)
iii. Standard vs. personalized. A standard and pre-programmed human motion analysis vs. personalized and self-adjusted for a specific person based on his/her own individual characteristics.

### Typical Human Motion Types and Recognition

These algorithms aim to determine and recognize the following normal or typical human motions or activities:
Sleeping
Walking
Running
Exercising
Talking (with hand movements)
Activity-High
Activity-Low
Normal (daily normal living activity—in homes, offices and elsewhere)

### Algorithms Based on Analytics, Formulae, Rules and Heuristics

These algorithms use rules, mathematical formulae, geometric equations, heuristics on the sensor signal data, absolute or relative, or its 1^{st} or 2^{nd} or 3^{rd} derivative signal data. Some may use Accelerometer (X, Y, Z acceleration data) only, some Accelerometer+gyro (rotation on X, Y, Z axes) (or) all 3: (Acc.+Gyro.+GPS).

### Pre-Processing:

There can be pre-processing step on the input sensor data to normalize, eliminate noise, clean up, adjust, and prepare for final algorithm processing.

### Analytics Algorithms

### Algorithm Method 1: Frequency-Domain Based

This class of algorithms transforms the time-domain sensor signal into frequency domain and then performs the analysis in the frequency domain. Some examples of such transformations could be Discrete Fourier Transform (DFT), Discrete Cosine Transform (DCT), Short-term Fourier Transform (STFT), Wavelet transforms, and others. The motivating idea behind such transforms is that some features of the motion pattern can more naturally be represented in the frequency domain and hence can be detected better. Once the signal has been transformed into the frequency domain, features can be extracted and used for analysis. For e.g. in the DFT, one may choose the magnitude of specific coefficients, in the wavelet transform one may compute the energy associated with certain sub-bands, etc. The final analytics detection/classification can be either some rule/heuristic based (such as thresholds), or machine learning based, or any combination thereof. Also, the system may choose to give a probabilistic output (a confidence value between 0 and 1) instead of a binary decision.

### Algorithm Method 2: Time-Domain Based

This class of algorithms utilizes computational techniques that operate on the time-domain signal data itself. For example a histogram of the amplitudes of the signal could be computed. Similarly other techniques in this class would include those that compute come salient features in the signal and use them for analysis, for e.g. the peaks and valleys of the waveform, zero-crossings of the waveform, etc. Other techniques in this class could include those that compute some statistical information about the signal (in a window of time), such as histogram, min/max, mean, variance or other higher order moments.

Derivative-based methods also fall into this category. For e.g. the first order derivative based methods for the accelerometer sensor represents the “jerk” of the signal and can be useful for certain kinds of motion. In the gyro sensor, derivative based techniques would compute the angular velocity and acceleration. Higher-order derivatives of any of these signals could be computed but due to the digital nature of data, they may turn out to be noisy.

### Algorithm Method 3: Modeling-Based

Sometimes signals can be more efficiently represented by a mathematical model. For example we could use stochastic models like the Autoregressive Moving Average Model (ARMA) models wherein the signal is modeled as the response of a linear shift-invariant filter to unit variance white noise. If we choose to model the signal as deterministic, then we could choose models such as the Autocorrelation method, the Covariance method, and others. A natural consequence of such modeling techniques is that it also allows the system to efficiently represent the signal by using only a few model parameters, thus allowing efficient storage.

### Algorithm Method 4:

These use absolute or relative data, 1^{st} or 2^{nd} derivatives, thresholds, min-max limits, time analysis, difference in peaks or average, etc. . . . .

Suppose we have N human actions that we want to recognize. We will create N analytic based actions classifiers which will classify any signal into 2 classes, one class is kth action and other class is any other movement. A confidence measure will be returned from each analytical classifier. If an action is classified into multiple motion types, the confidence measure will be used to choose the best of them.

We also have another alternative method, if the motion is classified into multiple motion types. We will create analytic classifiers for each pair motion type. So there will be N choose 2 such analytical classifier and this also gives a confidence measure of classification. This confidence measure will be added up for each of the conflicting motion types for an action. The motion type with greatest confidence will be chosen.

We choose a time interval which we call as unit time. Unit time can be 1 to 30 seconds long. Within this time interval we calculate various statistics of the Accelerometer+gyro signal+GPS. The statistics include mean value, standard deviation. Mean is subtracted from the signal and then monotonic segments are evaluated. Monotonic segments are time intervals where signal is either continuously increasing or decreasing. These segments will be stored in a vector. For each segment minimum and maximum value of the signal is calculated. Lengths of a monotonic segment will be the difference of maximum and minimum value of the segment. Those with very small minimum and maximum value are discarded as they can be noise. For each of the unit time we then store the number of monotonic segments and calculate the mean and standard deviation of the lengths of these monotonic segments. A number of other statistics are evaluated over the unit time average and standard deviation of 1^{st} and 2^{nd} derivative, number of +ve peaks and −ve peaks. Based on these statistics we will classify a unit time into 2 classes. We will use threshold which can be constant or dynamically varying for this classification. A confidence measure is also calculated using the statistics evaluated for the unit time and the threshold values. At each instance of time when we have a new signal value, we will do this classification over the unit time since current time instance. There will be N such classifications for N motion types. If the unit time is classified into multiple motion types, the motion type with highest confidence is chosen.

We choose a classification time interval which can be 30 sec to few minutes over which we will have a classification list for each time instance.

Suppose we have T time instance in classification time interval. We will now analyze these T classifications to make a decision if a motion type has occurred or not. We will do binning of motion types to find which motion type occurred most frequently. The confidence of this motion type is calculated as the ratio of frequency of the most frequently occurring motion and frequency of all other motions. A threshold is also used on the minimum possible value of frequency of most frequently occurring motion.

Some signals can have a global characteristic which is visible over classification time interval (30 sec to few minutes). Like sleeping can cause no motion for prolonged period of time, walking or running can cause continuous change in GPS values along with high frequency motion of hands etc. These characteristics may or may not exist. We analyze such motion type specific characteristic for each motion type. These characteristic are then used as a verification step for the motion type recognized over classification time.

### Algorithm Method 5:

This approach is similar to Algorithm Method 1. In this method we use a different approach to classify the unit time into motion types. Instead of one strong analytical classifier, we use several weak analytical classifiers. A weak analytical classifier is a classifier with very high true detection rate (˜99.9%) and false detection of the order 40-60%. We use many such weak classifiers in linearly thus achieving high true detection and very low false detection.

Each weak analytical classifier evaluates some feature over unit time for which a threshold is used to classify. We will explain some such features. All features and statistics used in Algorithm Method 1 can also be used here. We will explain more features analyzing the shape of the signal. We create a weighted histogram of the gradients of the signal in unit time. We bin the gradients in B bins. We use the weighted histogram of gradients for creating a weak analytical classifier. We find number of point of inflections in the signal in unit range. These are points where the curve changes from concave upward to concave downwards. Some actions can have very specific shape features like a sudden jerky movement can create a shark and high peak, waving of hands can create a periodic motion, sleeping can cause a long constant signals etc. We analyze the signal for such unique characteristics and call them contextual features. We use these contextual features as weak analytic classifiers.

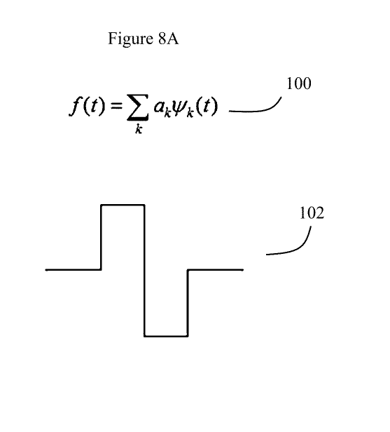

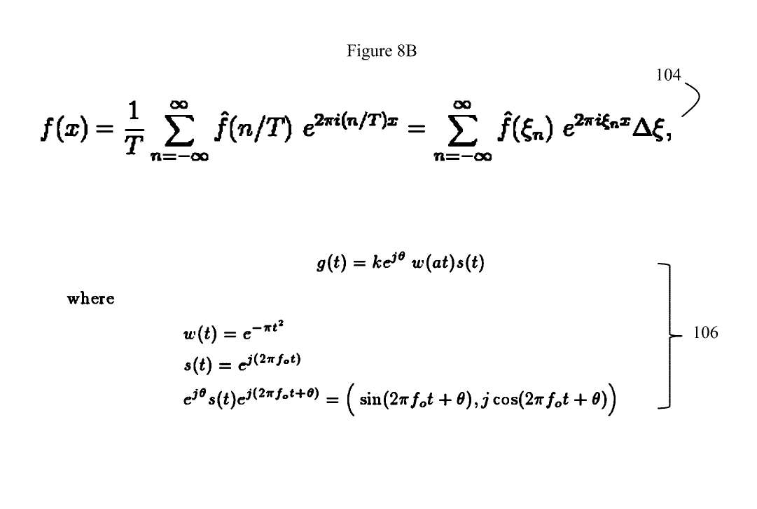

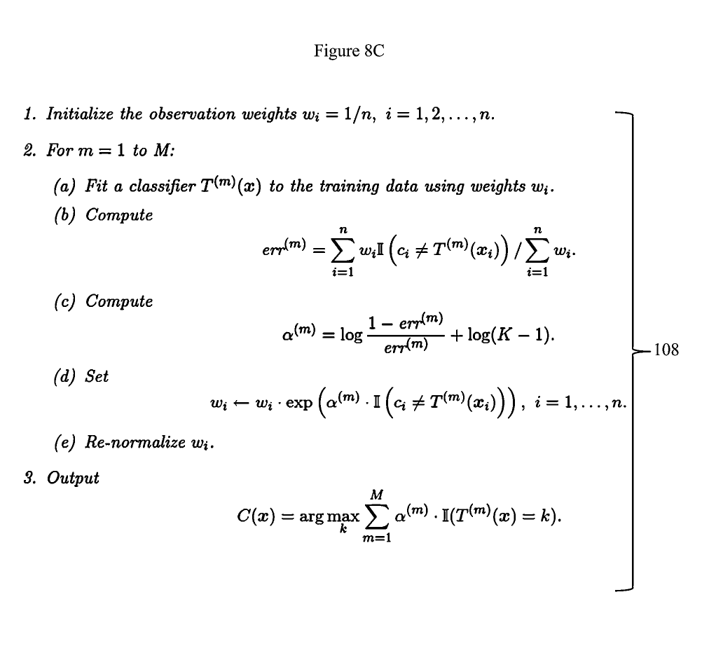

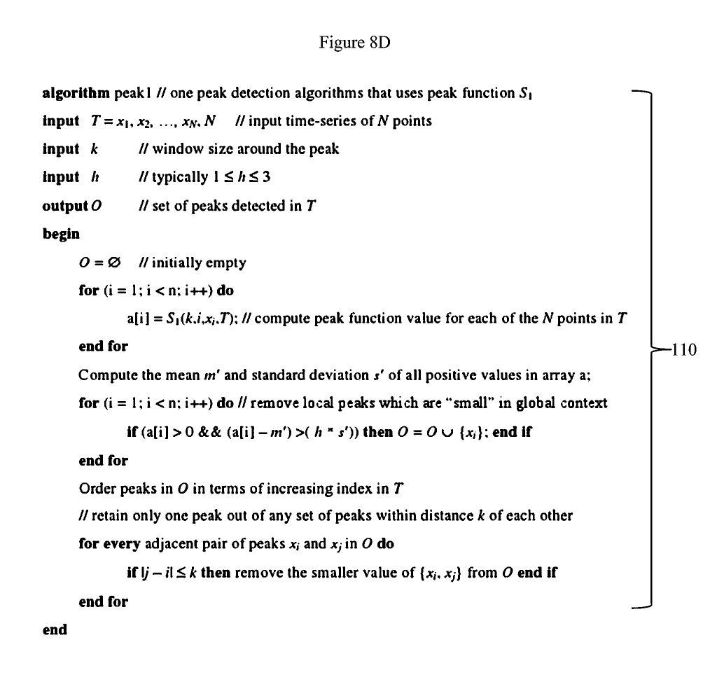

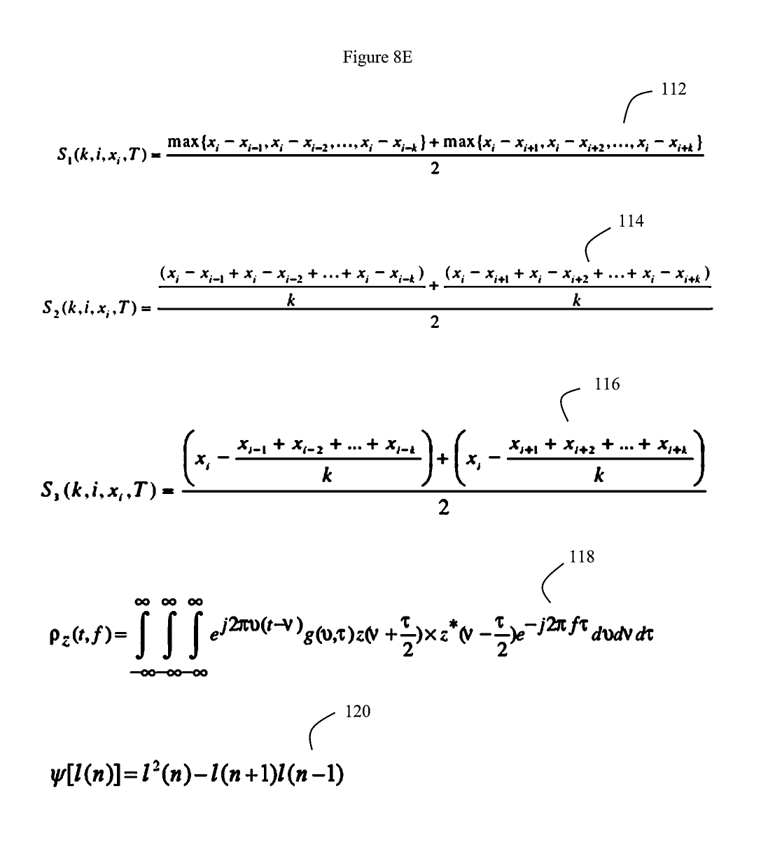

We use discrete Haar wavelets over unit time of signal and the resultant output is used to create weak analytic classifiers. Wavelets can be defined by following equation shown in FIG. 8A (100). FIG. 8A (102) shows an example of 1D Haar wavelets.

We do Fourier transform of the signal for different frequencies in unit time and analyze the resultant signal for average and deviation. The frequencies are chosen empirically for each motion type. FIG. 8B (104) shows the equation for Fourier transform

The statistics evaluated from the transform are then used to create weak analytic classifier. We use various other transforms to analyze the signal in time-frequency domain like 1-dimensional Gabor filters and then evaluate resultant signal with similar features as we used on original signal to create weak classifiers. Gabor filters are defined as shown in FIG. 8B (106):

Once the ensemble of weak classifiers is created, the resultant output classifier will give us N strong analytical classifiers for each of N motion types. We build up the confidence for a classifier while moving along each of the weak analytical classifier.

The rest of the algorithm is similar to the Algorithm Method 1 where list of classifications at unit times is used to classify the signal over a classification time.

Other algorithm types, such as feature or shape analysis algorithms may also be used.

### Algorithm Method 6: Hybrid Methods

In the above sections we have described different categories of computational techniques that could be used to derive analytical intelligence about the signal. However it does not preclude the possibility of using any combination of such techniques in an intelligent fashion before making the final decision. We may choose to hybridize the above techniques in a variety of ways, such as simple voting, or combining them probabilistically.

### Output

The outputs of these algorithms are:
Type: Motion & action recognition/classification: Type of human motion or action
Duration: Time—Start & end time of the motion and action
Intensity: Some measure of body movement or intensity level (0-100) to correlate to energy spent or calories burned
   Short Vs. Longer Term Motion & Action Analytics

### Short Term:

This class of algorithms infers analytic decisions by analyzing the signal over a short period of time, a few seconds to minutes. This may be necessary for 2 reasons—first, the memory storage capacity on the device may be very limited, and second, some applications may require immediate response time such as seizure detection and alerting, or continuously monitoring calories burnt for health and fitness applications. Hence the techniques in this category analyze the data in a short window of time and extract “primitive” intelligence. For e.g. in health and fitness applications the algorithm would compute the calories burnt every minute. In seizure detection application the algorithm would look for seizure-like pattern in the data by analyzing the signal in short time windows.

Any of the techniques mentioned in the sections above can be used here, as long as it is capable of working with a short duration signal. For e.g. simple methods such as amplitude based, as well as more complicated methods such as mathematical modeling based or frequency domain based methods could be used.

### Longer Term:

The analysis results from short-term analysis, referred to as “primitive” above can be aggregated together to form the long-term analysis results. The type of aggregation depends upon the algorithm and the application where it is used. For e.g. in the health and fitness monitoring application where the primitives (calories burnt every minute), could be summed up to compute the calories burnt for the duration of the exercise session. Similarly other statistics such as average calories burnt per minute and min/max calories burnt per minute could also be computed. Another example of aggregation could be computing the dominant pattern or trend in a collection of events. Consider the case wherein a care provider for an epileptic seizure patient is interested in finding out when during the day, frequency during the week and type of most frequent motion pattern that occurs for the patient. In this case the primitives computed by the short-term analysis algorithms would be the detection of any seizure event, and its time, duration and type. This information will be fed to the long-term analysis engine as and when each individual event happens. The long-term analysis would then do a statistical analysis to compute the information of interest.

Standard vs. Personalized

### Standard/Generic Algorithms:

In most cases, the algorithms mentioned above are supposed to be standard or generic and can be used for most subjects within the scope of the application. The parameters of the algorithm are selected to achieve a desired operating accuracy, such as a desired trade-off between true positive and false positive on the ROC. This can be achieved by evaluating the algorithm on a large dataset obtained from many subjects, and then tuning the parameters such that the desired operating point within a tolerance is achieved.

### Customized & Personalized Algorithms:

In some cases, customization of the generic algorithms maybe required for certain subjects. This may happen if the signal pattern obtained by the sensor is significantly different from the typical expected pattern. The system may compute certain test metrics and statistics on the data to decide whether the signal lies within the acceptable range (and hence standard algorithms may be used), or whether customized algorithm needs to be used. The customization can be done in several ways and is dependent on the algorithms being used. If a learning based algorithm is used, then the learning needs to be redone or adjusted using the samples obtained from the subject in a “calibration” phase. In rule or heuristic based systems, the decision rules can be adapted by adjusting parameters and thresholds based on the data obtained during the calibration phase. This adaptation or learning can be one-time or continuous and in some cases can be passive, i.e. the system does not need to go into the calibration phase to teach the system.

As an example, consider the case where a user is interested in alerting/recording only certain kinds of abnormal events for which no standard detection system is available. In this case, during the calibration phase the user will enact the behavior of interest and ask the system to learn it as an abnormal event. The system will then treat this signal data as a “positive” sample and other signals as “negative” samples. Now it can use machine learning or rule/heuristics based techniques to customize the detection algorithm.

### Algorithms Based on Learning and Classification

These algorithms use machine learning and classification techniques as follows, on the sensor signal data, absolute or relative, or its 1^{st} or 2^{nd} or 3^{rd} derivative signal data. Some may use Accelerometer (X, Y, Z acceleration data) only, some Accelerometer+gyro (rotation on X, Y, Z axes) (or) all 3: (Acc.+Gyro.+GPS).

### Pre-Processing:

There can be pre-processing step on the input sensor data to normalize, eliminate noise, clean up, adjust, and prepare for final algorithm processing.

### Algorithm Method 1: Supervised Learning Based

In supervised learning based methods, the system is presented with labeled “positive” and “negative” samples and a training algorithm is used to learn a classifier which can classify the data accordingly. The input to the classification algorithm can be the raw signal data itself or some transformation/mapping of the raw data. The learning algorithm could be Bayesian methods, Logistic regression, Neural Networks, Support Vector Machines, Hidden Markov Models, Decision Trees, Boosting methods etc. This binary classification can be extended to multi-class classification problem using techniques such as one-vs-rest.

### Algorithm Method 2: Semi or Unsupervised Learning Based

In some problems, labeled training data do not exist or is very hard to get. In such cases, a purely supervised learning method cannot be used. Hence a semi or unsupervised methods are required such as clustering techniques, wherein a natural partitioning of the data is to be inferred given the unlabeled samples. For e.g. consider the case where it desired to find out the different types of epileptic seizure patterns that a patient has over a period of time. This knowledge is not directly available and furthermore is hard to monitor a patient continuously over an extended period of time. But with the help of clustering techniques such as k-means or expectation-maximization this information can be obtained.

We could use such techniques to classify different types of activity of a person over a period of time, without having to explicitly categorize them into labeled activities such as sitting, running, lying, biking, walking etc. Signal data obtained from a sensor such as an accelerometer could be used to cluster (either directly using raw data or transformed into another feature space) into different types of activities.

### Algorithm Method 3:

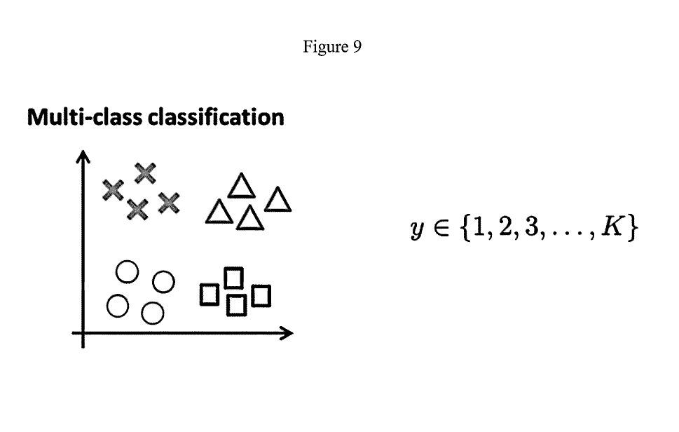

In this approach we build classifiers using machine learning methods. We build a multi-class classifier based on SVM such that there are N+1 classes for N motion types and the (N+1)th class is for any other action. FIG. 9 shows a drawing of a type of multi-class classification.

We use either linear classification or the non-linear classification using the kernel trick. We define the classification time segment which is the time interval over which an action is classified. The time segment can be 30 seconds to several minutes long. There are several feature sets that we use for training the classifier.
1. We divide the classification time segment into S overlapping segments. For each segment we calculate the histogram of gradients with B bins. We then create a feature vector of length S*B. This feature vector is then used as an input for learning SVM classifier.
2. We use a predefined set of 1D wavelet to create a feature vector over classification time segment.
3. We use Fourier transform to convert the signal into frequency domain. PCA is used to reduce the dimensions of the signal. The signal with reduced dimensions is used as the input feature vector for training SVM.
4. Divide the classification time segment into S overlapping time segments. For each segment calculate the number of monotonic segments. The array of the count of monotonic segments for a classification time segment is used as the input feature vector to train SVM.
5. For each time sub-segment, moments are calculated for zero, 1^{st} and 2^{nd} order. These moments are for all sub segments of classification time segment are used as input feature vector to SVM.

The classification technique is not limited to SVM. We can use neural networks, hidden Markov model, K-nearest neighbor, decision trees and random forests.

### Algorithm Method 4:

We use multi-class Adaboost to classify motion types over a classification time segment. A cascade of classifiers is build where each stage of cascade is a weak classifier. The weak classifier has the property that it has very high true detection rate (˜99.9%) and low false detection rate (40-50%). We use multi-class weak classifier that assigns a class label to an input action. We use decision trees for as our weak classifier. Summary of multi class Ad boost algorithm is given as shown in FIG. 8C (108).

### Others and Hybrid Algorithms:

A combination of supervised, semi-supervised or unsupervised methods can be used here.

These use learning methods (like Neural Nets, SVM, Bayes net, HMM, supervised or unsupervised learning, hybrids) can be used as well).

### Output

The outputs of these algorithms are:
Motion & action recognition/classification: Type of human motion or action
Time—Start & end time of the motion and action
Some measure of body movement or intensity level (0-100) to correlate to energy spent or calories burnt
Establish baselines for various activities—these can be
   Short Vs. Longer Term Motion & Action Analytics (Using Learning Based)
   Same as before (explained earlier)
   Standard Vs. Personalized (Using Learning Based)
   Same as before (explained earlier)

### 1. Motion Intensity, Energy Expended

The invention also covers methods and algorithms to determine:
The intensity of the motion or action and duration
Energy expended and calories spent (approximately)

The techniques used fall into one of these 3 categories:

### A: Approximate—Table Based

These types of techniques could use a pre-determined mapping rule based on inputs from various sensors. For e.g. knowing the type of activity the person is performing and the time duration, and information such as gender, age, height, weight etc. a table could be used to approximately determine the calories burnt by the person.

### B: Accurate—Formula Based

More precise computation of energy expended could be obtained by using the sensor data as described below. The acceleration data could be integrated to obtain velocity (assuming subject starting from rest) and the velocity can in turn be integrated to obtain distance. Note that by knowing the orientation information from gyro sensor, we can get better physical acceleration values which will lead to better estimates of velocity and distance. Alternatively for distance computation, GPS sensor information could also be used. Now knowing the distance moved and the time duration, and utilizing person specific information such as age, height, gender, weight etc. a regression analysis (learnt using training data) could be used to estimate the calories burnt. This regression formula could be linear or non-linear

This intensity and duration are calculated mathematically from the sensor data. Based on this information, energy spent is derived as follows. The movement/motion acceleration and angle sensor data are used to compute the velocity and distance of movement (by hand or body parts). Then integration if the motion curve will provide velocity and distance moved and it is used for energy spent.

### Baselines and Standards

The algorithms establish baselines—

A: standard across groups of people
based on age,
gender,
medical condition
Race or background etc.) or
   B: personalized/customized for the individual.

User can personalize and customize the pattern, thresholds and recognition based on personal movement or activity patterns. If there is no personalized recognition, then system can use the standard recognition on an average (or) based on the above factors by age, gender, medical condition or race etc.

That is the key behind our analytics.

### 2. Wired and Wireless Connectivity

The device communicates with the outside world in one of the 2 ways:

### 1: Wireless

There are various types of possible short and long range wireless connections. These include

A: Bluetooth and Wifi joint wireless. There are chips that jointly do both or separately provide Bluetooth and Wifi 802.11 access. This provides connection to mobile phone or home Wifi network.

The device can send data to either of these.

B: Single local wireless Bluetooth only—will only go to the Mobile phone with Bluetooth ability
Wifi only—can connect to either phone or home Wi-Fi router
C: 2G/3G/4G wireless. This uses cellular or GSM communication to the remote cell/mobile networks provided by several network carriers

### 2: Wired

Wired connection is also possible via USB/Serial connection and/or Ethernet. These require a different chip set and drivers in the device. But the device can be made to use wired connection for certain markets or hospitals or where wired connections are preferred and available. Examples of this type of connectivity are shown in FIG. 5.

### 3. System Architecture, Components & Connectivity

There are 3 major system components or devices:
1. Body worn device (under discussion and illustration here)
2. A mobile phone (a typical smart phone)—Optional
3. A remote Computer or server (typically on the Internet on WAN or a local server on LAN)

The body worn device connects either to the mobile phone (or) the server on the Internet (or local). There are 2 models for the device one with Bluetooth and/or Wifi and other with 3G/4G modem to connect directly via the cell/GSM networks.

In the system architecture type 1: the device (body worn) connects to the mobile smart phone and which in turn connects to the internet or cloud server

In the System architecture type 2: The device (body worn) connects to the internet/cloud server directly via 2G/3G/4G modem to the internet. An example of this type of overall system is shown in FIG. 6.

The architecture will support and enable 3 types of processing or action:

1: the body worn device will make either make an internal decision and send the summary, results, alerts and data out to the (i) phone and/or (ii) to the internet server
2: (or) alternative, the device will send the data out to the phone and the data processing fully or partly is done by the phone
3: or the data is sent to the cloud or internet server or a PC, (from the body worn device) or the phone, then the data is processed outside in the Internet or PC.

While we expect in many cases, the data can be processed mostly in the device, the invention and architecture will fully enable and support processing the data in the phone or on the cloud.

All the same algorithms and strategies and methodologies can be executed inside the device or on the outside CPU or computer (be it either on the smart phone or a server computer).

Hybrid processing is also possible, where some processing or computation is done inside the device, and rest or remaining processing is done outside the device (be it either on the smart phone or a server computer).

### 4. Abnormal and Deviation Detection and Alerts

The document and methodology also covers another aspect of recognition, detection and alerting. The system can determine abnormal or behavior from the normal. Any deviation the normal sensor or pattern can be treated as abnormal and can be output as an abnormal or deviation event. Then that event can be sent out as alert. The Abnormal behavior or motion or action can be determined by:
Threshold or level of the sensor data
Or Following a rule or logic on the sensor data or level
Or condition of the body motion or based on sensor data
Or action by time or intensity of the body action
Or Learning based deviation or abnormal detection
Or simply by deviation from normalcy

System can find and know what is typical or normal. Any deviation from the normal pattern can be tagged or determined as abnormal or deviation. Then these are output as events or messages from the body device. These messages or alerts or alarms are output from the device to the mobile phone or the internet cloud via the above mentioned wired or wireless connectivity methods.

### 5. Notification & Alerts

System or device has the ability to sent notification, messages or alerts. There are 3 types of data that the device can output or notify or send out:

Type 1 output data: Events or alerts or messages

These are specific events or message such as human body action or recognized activities. These can also be abnormal or deviation events or messages from the normalcy.

Type 2 output data: Normal motion and summary of daily or hourly or weekly or monthly or yearly motion or action or human daily routine summary or reports or activities. These tend to be action or motion names, times, duration intensity, energy expended, patterns, some annotation, and determination of the summary of these algorithms or recognition

Type 3 output data: are raw sensor or processed sensor data. These can be accelerometer, gyro, or GPS and other sensor data. These data can be output as is or processed and reduced or compressed or uncompressed or combined or summarized in some form or list of data or table or database etc.

Alerts or message are specific alarm or summary for other to take action on. This can be an abnormal event and someone needs help. Or someone is exercising too long or sleeping too long. These messages or events are logged in the device also sent out.

These notification or messages or alert can sent out in one of these 3 ways:

1: Locally to the mobile phone via Bluetooth or Wi-Fi
2: to the remote server or PC, or Internet or Cloud—directly via wireless or via the mobile phone.
3: Via the wired connection to the local PC or server

Alert or notification can be sent by any one of the entities:
Body worn device
Mobile phone
Internet/cloud server
PC/Connected server

Notification can be an event that is logged or a message or SMS or Email that is created from the notification or alert. These following forms of alerts and notification are made possible:
SMS—Simple text message
MMS—Multimedia
Email
Pop up message or notification
Phone call
Log an entry into a file or report or database
An posting on a social site or web site or blog
A message via Twitter or Facebook—messaging methods

### 6. Trending

Trending is about analyzing the patterns and movements over a period of time. The system both inside the body worn device; and/or in the mobile phone; and/or on the internet server, can continue to analyze the body motions from recent past to before or long time back. These movements are compared and analyzed for the variations. These variations can be shorter or longer durations, smaller or higher intensity, shape patterns, movement sequence patterns, styles, gaits, etc., can be compared and analyzed. The trends can be neutral or positive or negative. These trends as same or similar to: comparison of sensor signal over time and motion/action patterns over time, —will create trending summary. These trend summaries are reported in the analytics or “trending” of the human motion or the user data over time. These can be reported, summarized or documented or annotated in either the device, or in the mobile phone or in the internet cloud server in the summary reports.

### 7. Reporting, Summary, Data Types

The device or the processing unit will create the following reports, summary & Data Types
Sensor raw data
Sensor Processed and summarized data
Processed analytics and actions recognized and results
Resulting summary, time, actions, intensities, energy, human motion summary
Alerts, Notification, alarms, messages
Trends, variations over time
Overall summary and report in data format or HTML format
Overall summary and report in PDF or tabular or words formats

These reports and summary can be created by one of these:
Body worn device
Mobile phone
Internet/cloud server

### 8. Data Flow, Data Storage, Algorithm Locations, and Alert Flow

Data flow typically happens as follows.
From Sensor
Temporarily hold in memory
To CPU/Processor
More processed results in memory
Communication outside
Reports and alerts to outside

Examples of this type of data flow are shown in FIG. 7.

### Data Storage

Data storage location can be one or more of the following:

### Device:

Local RAM temporary memory from Sensor
Local RAM or NAND temp or perm memory for results and summary data
Local Firmware perm memory for program and execution data
Sensor data over time, and results data and trending—stored in device (optional)
Alerts, notification, reports and summary

### Mobile

Sensor and summary and results data and trending—stored in mobile phone (optional)
Alerts, notification, reports and summary

### Internet/Cloud/External PCs/Server

Sensor and summary and results data and trending—stored in mobile phone (optional)
Alerts, notification, reports and summary

### Several Embodiments and Forms

The whole invention can be applied to all 3-4 forms of applications or utilities or solutions:
1. A whole system. An independent and self-functioning system (including hardware, firmware, sensors, software and algorithms)
2. A method or methodology, for making technical product and making a viable solution or component
3. Algorithms—primarily the software, math processing and getting detection, recognition and alerting to work and the method to do the software
4. A module with one of more of these method or algorithm or a system component. (including any/some hardware, firmware, sensors, software and algorithms)

Embodiments of these above forms are viable and possible and presented in this invention. A utility patent can be followed or filed based on any of the above embodiments or forms.

### Body Fall Detection, Recognition and Algorithms

### 9. Abnormal Motion and Fall Detection & Alerting

This documents all deviations from the normalcy as abnormal patterns or movements or body motion. These can be:
Falling down
Specific types of complex partial seizures (like Myoclinic, Atonic, Jerks, Convulsions, Hand, leg, motion movement abnormalities)
Tremors (like in Parkinson diseases)
Fighting or defense, kicking, punching etc.

The document and methodology also covers another aspect of recognition, detection and alerting. The system can determine abnormal or behavior from the normal. Any deviation the normal sensor or pattern can be treated as abnormal and can be output as an abnormal or deviation event. Then that event can be sent out as alert. The Abnormal behavior or motion or action can be determined by:
Threshold or level of the sensor data
Or Following a rule or logic on the sensor data or level
Or condition of the body motion or based on sensor data
Or action by time or intensity of the body action
Or Learning based deviation or abnormal detection
Or simply by deviation from normalcy

System can find and know what is typical or normal. Any deviation from the normal pattern can be tagged or determined as abnormal or deviation. Then these are output as events or messages from the body device. These messages or alerts or alarms are output from the device to the mobile phone or the internet cloud via the above mentioned wired or wireless connectivity methods.

### Body Fall Detection& Other Abnormal Movement Detection

### Analytic Based Algorithm

### Algorithm 1

We will create an analytics based classifier to classify the motion type of fall detection. We have to do a binary classification for the incoming sensor data from accelerometer to classify as fall or not. We choose a time interval which we call as unit time. Unit time here can be 0.1 to 2 seconds long. Within this time interval we calculate various statistics of the Accelerometer. The statistics include mean value, standard deviation, time average and standard deviation of 1^{st} and 2^{nd} derivative, number of +ve peaks and −ve peaks, amplitude of the peaks. We also calculate the relative motion, speed and acceleration at each time instance. We use threshold which can be constant or dynamically varying for this classification. For fall there would be sudden movement in accelerometer and thereafter there can be some small movement or no movements all.

We choose a classification time interval which can be 1-4 seconds over which we will have a classification list for each time instance. Suppose we have T time instance in classification time interval. We will now analyse these T classifications to make a decision if a motion type has occurred or not. We will do binning of motion types to find which motion type occurred most frequently. The confidence of this motion type is calculated as the ratio of frequency of the most frequently occurring motion and frequency of all other motions. A threshold is also used on the minimum possible value of frequency of most frequently occurring motion.

### Algorithm 2

We model fall detection as a sudden movement which is preceded and followed by relatively less movements. There would be a spike in the movement in a small interval of time which we can take as 0.1 to 1 seconds which we will call as fall time interval. There will be benign time intervals before and after fall time interval in which there can be any usual motion but this motion will be overall less as compared to the motion in fall time interval.

We define the problem statement as detection of motion peak which are preceded and followed by benign motion (benign motion is general day to day motion).

We will run the fall detection algorithm on all 3 signals of X, Y, Z from accelerometer and have a confidence value from the three detections. We will then combine the confidence values to make the final decision. We start by detecting all peaks in the signal data using following algorithm as shown in FIG. 8D (110).

There are several different ways to compute S which captures the spikiness of the point xi in local context.
1. For a given point xi in T, the following function S1 computes the average of (i) the maximum among the signed distances of xi from its k left neighbors and (ii) the maximum among the signed distances of xi from its k right neighbors. Low values of k (e.g., 3 to 5) are usually suitable, if most peaks are “thin”. Values of S1(k, i, xi, T) indicate the “significance” of the height of the peak at the ith time instant, as shown in FIG. 8E (112).
2. Function S2 computes the average of (i) the average of the signed distances of xi from its k left neighbors and (ii) the average of the signed distances of xi from its k right neighbors, as shown in FIG. 8E (114).
3. Function S3 computes the average signed distance of the ith value xi in T from the average value of its k temporal neighbors, as shown in FIG. 8E (116).

Using each of the above peak functions, we could easily write an algorithm to detect all peaks in the given time-series T. We show below the algorithm that uses the peak function S1 (other peak detection algorithms are very similar, except that each uses a different peak function). The peak function S1 computes its value at each point using the local window (context) of size 2 k around that point. All points where the peak function has a positive value are candidate peaks. We rule out some of these locally detected peaks using the global context (time-series as a whole) as follows. We compute the mean and standard deviation of all positive values of the peak function and then retain only those points xi in the time-series which satisfy the condition as shown in peak algorithm where h is a user-specified constant.

Once all peaks are detected, we start discarding peaks which do not suite the criteria of a peak corresponding to a fall. We use peaks which are larger than a specific value which is user defined.

Then we take larger time intervals before and after the peaks called benign time intervals. We use several criteria to analyze these time interval to discard the false positives. We discard the peaks if there is another peak in the benign time interval. We measure the mean, minimum and maximum values and standard deviation in these time intervals. All these parameters will be relatively smaller in the benign time interval as compared to the time interval of the peak.

### Algorithm 3

We detect spikes using the time-frequency distribution of the signal. As spikes are short-time broadband events, their energy patterns are represented as ridges in the time-frequency domain. In this domain, the high instantaneous energy of the spikes makes them more distinguishable from the background. To detect spikes, the time-frequency distribution of the signal of interest is first enhanced to attenuate the noise. Two frequency slices of the enhanced time-frequency distribution are then extracted and subjected to the smoothed nonlinear energy operator (SNEO). Finally, the output of the SNEO is thresholded to localize the position of the spikes in the signal. The SNEO is employed to accentuate the spike signature in the extracted frequency slices. A spike is considered to exist in the time domain signal if the spike signature is detected at the same position in both frequency slices.

A time-frequency distribution (TFD) of a signal is a joint representation of both time and frequency domains of the signal. For a given signal, k(t), the TFD that belongs to the quadratic class can be expressed as shown in FIG. 8E (118):

Where z(t) is the analytic signal associated with x(t) and g(v, t) is a two-dimensional kernel that determines the characteristics of the TFD. By setting g(v,t)=1, for example, ρz(t, f) represents the Wigner-Ville distribution.

To detect spike events, the signal is mapped into the TF domain. In this domain, the TFD tends to spread the noise throughout the TF plane, hence reducing its effects on the useful part of the signal. To further attenuate the effects of noise on the TFD of the signal, the SVD-based technique. Once the TFD of the signal has been enhanced, two relatively high frequency slices are extracted (in the TFD, the frequency slices are extracted from higher frequency area). If both frequency slices have any signature of a spike at the same position, the related time domain signal is judged to contain a spike at that position. To further amplify these signatures, the NEO is applied to the frequency as shown in FIG. 8E (120).

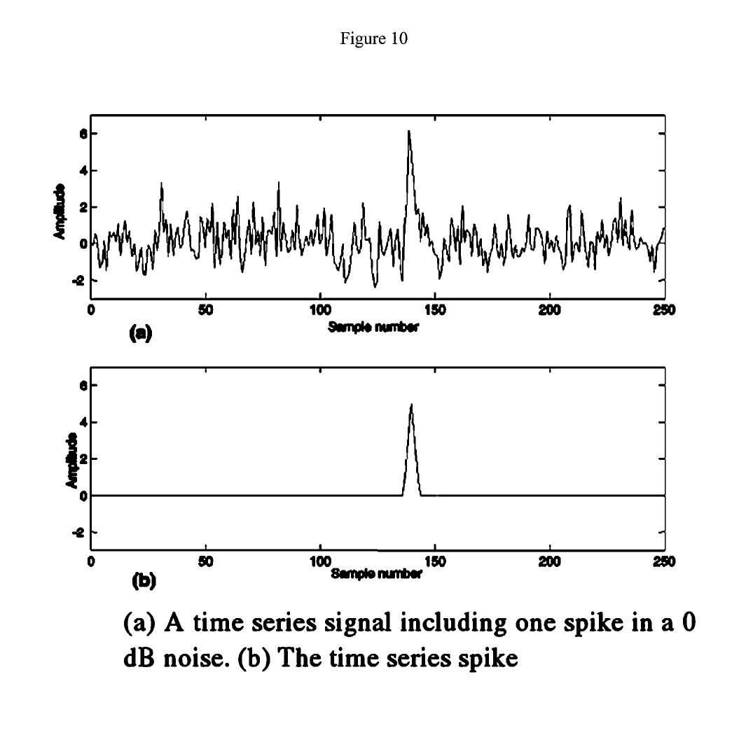

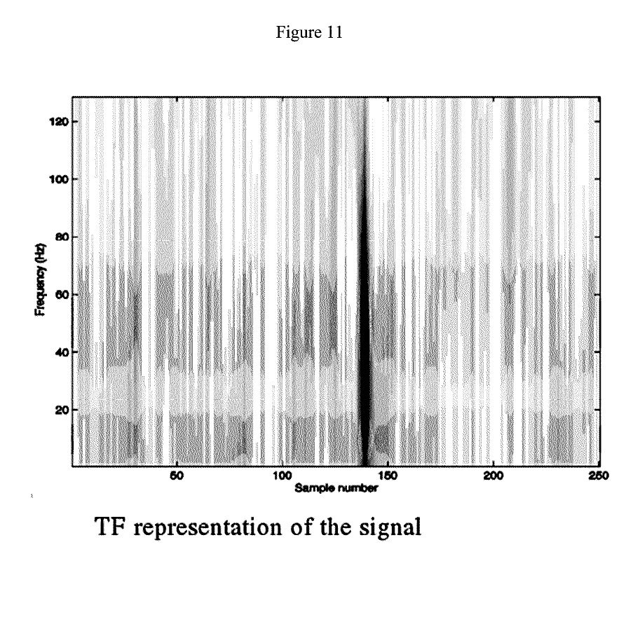

The local peaks at the output of the SNEO that are higher than a predefined threshold are considered as an indication of the existence of a spike at that location in the time-series. To detect spikes in a signal using the TF signal analysis techniques, a reduced interference distribution (RID) is needed in order to reduce the problem of cross-terms raised by the multicomponent nature of spikes. A diagram showing TFD analysis signals is shown in FIG. 10, and a diagram showing a TF representation of the signal is shown in FIG. 11.

### Learning Based Algorithm

In this approach we build classifiers using machine learning methods. We build a 2 class classifier based on SVM. We use either linear classification or the non-linear classification using the kernel trick. We define the classification time segment which is the time interval over which an action is classified. The time segment can be 1-4 seconds long. There are several feature sets that we use for training the classifier.
6. We divide the classification time segment into S overlapping segments. For each segment we calculate the difference of maximum and minimum value of the signal.
7. We divide the classification time segment into S overlapping segments. For each segment we calculate the maximum distance between the (X, Y, Z) points of the accelerometer data. For fall this value will give a sudden spike which can be learned by the classifier.
8. We divide the classification time segment into S overlapping segments. For each segment we calculate the histogram of gradients with B bins. We then create a feature vector of length S*B. This feature vector is then used as an input for learning SVM classifier.
9. We use a predefined set of 1D wavelet to create a feature vector over classification time segment.
10. We use Fourier transform to convert the signal into frequency domain. PCA is used to reduce the dimensions of the signal. The signal with reduced dimensions is used as the input feature vector for training SVM.
11. Divide the classification time segment into S overlapping time segments. For each segment calculate the number of monotonic segments. The array of the count of monotonic segments for a classification time segment is used as the input feature vector to train SVM.
12. For each time sub-segment, moments are calculated for zero, 1^{st} and 2^{nd} order. These moments are for all sub segments of classification time segment are used as input feature vector to SVM.

The classification technique is not limited to SVM. We can use neural networks, hidden Markov model, K-nearest neighbor, decision trees and random forests.

We also use Adaboost to create a cascade of weak classifiers from the feature set we have described above to classify fall from other motion types. The weak classifier has the property that it has very high true detection rate (˜99.9%) and low false detection rate (40-50%). Cascading many such weak classifiers gives a classifier with high detection accuracy and very low false detection rate.

### Abnormal Position and Fall Detection (Additional Algorithms)

### Body Fall Detection & Abnormal Movement Detection Algorithms—Additional

### Introduction

Fall detection is an important problem which finds uses in a variety of applications such as elderly care, device protection, etc. In this work we describe a fall detection system which utilizes tri-axis accelerometer as the primary sensor with other additional/optional sensors namely gyroscope, magnetometer and barometer.

Note that there could be more than one of each of the sensors worn at different locations on the body of the person.

### Fall Detection Algorithm

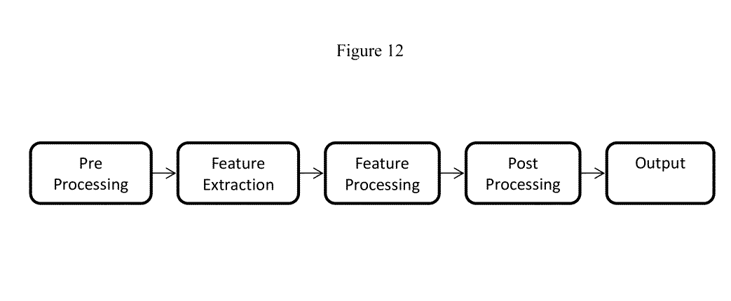

The overall design of the detection algorithm can be split into several stages as illustrated in FIG. 12. Note that some stages can be optional depending upon specifics of the analysis.

### PreProcessing

This is an optional stage where the raw sensor data is preprocessed so that it becomes more useful in the following stages of the algorithm. For example when the raw sensor data is noisy some noise-reducing filtering like low-pass filtering, median filtering or Kalman filtering may be required. Similarly, pre-processing may be necessary to change the sampling rate or the bandwidth of the signal if the downstream stages require so.

Here we describe how a Kalman filtering can be used to reduce noise. A process x(n) is assumed to follow a linear stochastic difference equation as follows

x(n)=Ax(n−1)+Bu(n−1)+w(n−1) (1)

The measurements or observations s(n) are formed as

s(n)=Hx(n)+v(n) (2)

Here w and v represent the process and measurement noise respectively, u represents the optional control input, and A, B, H are matrices which may or may not be constant. Using a “predict” and “update” mechanism, the Kalman filter iteratively corrects the estimate of the signal thus reducing the noise.

### Feature Extraction

This is the stage where several features of interest can be extracted from the sensor data which can then be used for fall detection. A variety of techniques can be used here as described below. Note that these measures could be computed either in time domain directly or the data could be first transformed into frequency domain (such as Discrete Fourier Transform) and the features extracted subsequently.

### Sliding Time Window Based

In this category of methods, the signal data is analyzed over a period of time (time window) and some measures (either directly or some transform of the signal) calculated based on that. The time window could be as small as few samples corresponding to a fraction of second, or several minutes depending upon the sampling rate and the memory resources available. Hence in this category of feature extraction methods, decisions are not taken based on the individual samples. Instead they are based on a collection of samples.

### Individual Samples Based

In this category of methods, the individual samples of the signal data (either directly or some transform) are used to compute the features. Each individual sample is considered as a potential feature candidate and some meaningful information extracted from it.

As an example we could estimate the vertical and horizontal component of user's motion from the 3D accelerometer signal and uses that as the feature. If the accelerometer sensor is constrained to be in a fixed orientation on the user's body then obtaining these components is straightforward. However, in most cases it is not possible to impose this restriction or assume this constraint. Hence a sensor-orientation independent method can be used, and in the following we describe an example of such a method. We can assume a constant acceleration due to gravity vector \vec{g} as (gₓ, g_{y}, g_{z}). Now let \vec{a}=(aₓ, a_{y}, a_{z}) be the 3D acceleration vector obtained by the sensor in a given sampling interval. The vector which corresponds to user's motion is thus given by \vec{m}=(mₓ, m_{y}, m_{z})=(aₓ−gₓ, a_{y}−g_{y}, a_{z}−g_{z}). The projection \vec{p}ᵥ of \vec{m} on to the vertical direction is thus given by

$${\vec{p}}_{v} = ( \frac{\vec{m} \cdot \vec{g}}{\vec{g} \cdot \vec{g}} ) \vec{g} \qquad ( 3 )$$

and the projection \vec{p}ₕ of \vec{m} on to the horizontal direction is given by

\vec{p}ₕ=\vec{m}−\vec{p}ᵥ (4)

Knowing these components we can get an estimate of the user's motion in the fall direction (\vec{p}ᵥ) and the user's horizontal motion (\vec{p}ₕ).

### Statistical Measures

This type of feature extraction strategy computes a statistic of the signal over a period of time, for example sum of squared amplitude (energy), area under the graph, mean, variance, standard deviation or other higher order statistics. It can also compute rank statistics such as min, max, median etc.

As an example a feature which encodes the standard deviation of the magnitude of the 3D acceleration vector (ā), computed in a time window is shown below

$$f = stddev ( \vert \overline{a} \vert ) \\ \vert \overline{a} \vert = \sqrt{{a}_{x}^{2} + {a}_{y}^{2} + {a}_{z}^{2}} \qquad ( 5 )$$

where aₓ, a_{y}, a_{z} are the components of the 3-axis acceleration of the sensor.

### Salient Points Measures

This type of methods locates some salient points in the signal data such as the peaks, troughs, zero-crossings etc. Then some measures can be computed based on these points, for example the count of such points in a given time window, the average gap between the salient points, the standard deviation of the gap between salient points etc.

As an example consider a feature which computes the tilt angle (τ) of the body as—

τ=cos⁻¹(a_{z}/g) (6)

where G is the magnitude of the acceleration due to gravity. This measure can be computed at each sampling interval and a peak detector could detect the maxima and minima within a time window. Having the peaks, we could compute the average value of the peaks, standard deviation of the peak values, average value of the gap between successive peaks, standard deviation of the gaps between peaks, and so on.

### Difference Measures

In these methods, some kind of differencing or comparison is used as the feature. For example derivative (first of higher order) based measures compute the difference between neighboring samples. Similarly, other comparison based measures could be used such as those that compute the angle difference between the 3D acceleration vectors. The differencing could be done between individual values of neighboring samples, or between some representative values of time windows.

For example a feature φ that encodes the difference between the 3D acceleration vectors at time point t1 and t2 denoted respectively by \vec{a}ₜ₁ and \vec{a}ₜ₂ could be—

φ=\vec{a}ₜ₁·\vec{a}ₜ₂/(|\vec{a}ₜ₁∥\vec{a}ₜ₂|) (7)

### Signal Modeling Based

This category of methods represents the signal (original or transformed) by means of a mathematical model and the coefficients or parameters of the model are used as the features. In the Auto Regressive Moving Average (ARMA) model, a signal s(n) can be modeled as the response of a linear shift-invariant filter to unit variance white noise. The ARMA model of order (p,q) is represented as follows—

$$H ( z ) = \frac{{B}_{q} ( z )}{{A}_{p} ( z )} = \frac{{\sum }_{k = 0}^{q} {b}_{q} ( k ) {z}^{- k}}{1 + {\sum }_{k = 1}^{p} {a}_{p} ( k ) {z}^{- k}} \qquad ( 8 )$$

Therefore a process s(n) can be modeled as an ARMA (p,q) process as the response of the above filter to a unit variance white noise. The coefficients b_{q} and aₚ thus form the feature representation of the original signal s(n).

### Transform Based

This category of methods transforms the original time domain signal into a purely frequency domain (such as Discrete Fourier Transform) or a frequency-time domain (such as the Discrete Wavelet Transform). Below we describe how a DWT can be used to extract features representing the original signal s(n). A given choice of mother wavelet ψ leads to a low-pass filter g and a high-pass filter h. The signal s(n) is decomposed into a low-pass component and a high-pass component by filtering it with g and h. The low-pass component forms the Approximation coefficients and the high-pass component forms the Detail component. These are the level 1 coefficients. The above filtering process can then be repeated on the low-pass component from this level, thus forming a multi-resolution wavelet coefficients representation of the signal s(n). These coefficients now form the features of the original time domain signal and they can be processed in the Feature processing stage to make decisions on the Fall event.

### Hybrid Methods

Any combination of the above methods could be used as a hybrid feature extraction method.

### Feature Processing

After the features have been extracted, they need to be processed or analyzed in order to exploit the information in them. In our context it means we have to process them in order to make a decision as to whether a fall event has occurred or not. Two types of approaches can be used as described below—

### Empirical Rules Based

Here the features are analyzed empirically to decide what decision rules to use in order to classify the event as fall or not. Each of the features computed above has a range that can be computed based on experimental data. A suitable threshold can then be chosen on each of the extracted features to decide between a fall event or otherwise.

For example we can detect the fall direction of the person and if the direction is within a threshold of the vertical direction we may classify it as a fall event, or further validate it using other features and decisions. Similarly, another example could be when the features of choice are the Discrete Wavelet Transform coefficients of the original s(n) and the coefficients in a particular band (s) could be thresholded to classify as a fall event.

### Machine Learning Based

Here the decision rule is learnt by employing a machine learning algorithm which learns a classification rule using a training data set. The training data set consists of class labeled samples, positive (fall) and negative (no fall) and the corresponding features extracted from the samples. Machine learning algorithms such as Logistic regression, Neural Networks, Support Vector Machines, Decision Trees, Boosting and others can be used to learn the optimal decision rule which separates the positive samples from negative samples.

Further, multi-class learning techniques can also be used to not only classify fall vs no-fall, but also distinguish between different kinds of fall event. For e.g. a fall event succeeded by a period of no motion could be differentiated from a fall followed by some movement. Similarly a fall event preceded by a state of rest could be differentiated from a fall preceded by a state of normal motion.

### PostProcessing

This is an optional stage wherein the output of the Feature Processing stage can be validated using some complimentary information from any of the sensors. For example a gyroscope sensor could provide the rotational rate of the body and this information could be used to validate the detection of fall event. For a dynamic transition of the posture during an unintentional fall event, the rotational rate of the body will be usually higher than normal and this fact can be utilized to minimize false detections. Similarly a barometric pressure sensor could detect a sudden rise in pressure as the person fell, thus validating the fall event.

### Several Embodiments and Forms for Fall and Abnormal Motion Analytics or Detection

The whole invention can be applied to all 3-4 forms of applications or utilities or solutions:
1. A whole system. An independent and self-functioning system (including hardware, firmware, sensors, software and algorithms)
2. A method or methodology, for making technical product and making a viable solution or component
3. Algorithms—primarily the software, math processing and getting detection, recognition and alerting to work and the method to do the software
4. A module with one of more of these method or algorithm or a system component. (including any/some hardware, firmware, sensors, software and algorithms)

Embodiments of these above forms are viable and possible and presented in this invention. A utility patent can be followed or filed based on any of the above embodiments or forms.

### Additional Abnormal Detection Algorithms

In some embodiments, some of the detection algorithms previously disclosed in U.S. patent application Ser. No. 12/154,085, the contents of which are incorporated herein by reference, may also be used either with or without various modifications.

For example, one way of detecting abnormal motion is to monitor the motion of one or more parts of the body of patient. During an abnormal activity there are rapid and jerky movements of one or more body parts, such as the hands, legs, torso, and head. Abnormal motion can be detected by measuring the change in output of a motion detector, the frequency of the change, and/or amplitude of the change indicating a movement of one or more body parts.

There are different types of motion detectors, which may be used of motion detector. One type of motion detector is an accelerometer, which measures acceleration. Ordinarily, when stationary, each part of the body experiences the acceleration of gravity (an accelerometer cannot tell the difference between a body being accelerated as a result of the body's changes in velocity and the body being pulled by a force, such as gravity). From the changes in acceleration, changes in position and/or velocity may be inferred. When a body part moves, the acceleration of the body part changes, and thus the change in the acceleration indicates a motion. Another type of sensor data is gyro sensor data. Gyro sensors may detect the rotation along X, Y, and/or Z axes. The rotation angles and position can also be used to detect motion and particular types of motion. While accelerometer measures linear axis changes, gyros measure the rotation changes.

The sensor that is used as motion detector may a small device that can fit into an enclosure the size of a wristwatch. The motion data may be measured in two-dimensions (e.g., along two perpendicular axes, which may be referred to as the X and Y axes) and/or three-dimensions (e.g., along three perpendicular axes, which may be referred to as the X, Y, and Z axes). Acceleration, frequency, and amplitude (angle, angular velocity, angular acceleration, and angular impulse or jerk) values above a certain threshold are indicative of abnormal body movements that occur during an abnormal motion. If two two-dimensional (X, Y) motion sensors are used as motion detector, each of the two-dimensional (X, Y) motion sensors may be paced along perpendicular axes. The two-dimensional and/or three-dimensional motion sensors can be useful to detect the jerky and back and forth movements. Additional detection algorithms are discussed below. The overall device can be positioned and/or mounted on the patient's body.

Abnormal alert system alerts a concerned party when a seizure occurs. Seizure alert system may be a PC, laptop, PDA, mobile phone bell, and/or other unit capable of indicating an alert. Information in signals from cameras and/or device are analyzed, and if it is determined that an abnormal motion is occurring, an alert is output from alert system. Motion alert system may include a monitor for displaying seizure alerts and/or for displaying motion data. In an embodiment, abnormal motion alert system may include a processor for analyzing the signals from cameras and/or motion detector. In an embodiment, system is a general purpose alerting system and can also alert other motion disorders.

Motion sensors that are included within motion detector may be attached to the wrist explicitly capture the motion along the x, y, and z directions. The data obtained may be a time sequence of the instantaneous acceleration experienced by the sensor. Several approaches can be used here to detect any abnormal seizure-like activities. These approaches can be divided broadly into two categories:

In an embodiment, a processor, which may be located in abnormal motion alert system or elsewhere may run algorithms to determine whether a seizure is occurring, and when it is determined that a seizure is occurring alerts may be output from motion alert system. In an embodiment, along with the alerts, one or more confirmation images and/or accelerometer or other motion sensor plots may also be sent to a concerned party, such as a doctor and/or other caregiver. The alert may be sent, via SMS, MMS, email, IM, or WAP or other message protocol. The alert may include an alert message, alarm signal, beeps, local in-device sound alerts, and/or alarms, which may be produced by a smart phone, mobile phone, or other device, which may have built in alarms and alerts. A Receiver receives signals from transmitters on wireless units such as motion detectors.

One example of an embodiment encompassed may include input sensors (video and/or motion sensors, such as accelerometers), one or more computers for receiving and processing data from the sensors, connectivity interfaces, and a system for remotely monitoring and for alerting (e.g., sending an alarm) a concerned party. The connectivity interface may include any of a number of communications interfaces, such as Bluetooth interfaces and/or Wifi, wireless interfaces, and/or wired interfaces, which may use IP/LAN connections and/or serial port connections, such as USB.

In an embodiment, device (with accelerometer/gyros) sensor may be used to determine whether there is an abnormal motion. This embodiment may include features and elements of both video and accelerometer/gyro sensor systems, mentioned above. Both detectors may run in parallel. Images from the video system may be used for additional confirmation and validation of the data from the accelerometer systems. These systems may run 24 hours per day, seven days a week, 365 days per year, all the time, or on an as-needed basis. The abnormal motion system, alert system, motion detector may be powered externally or may be powered by a battery. In an embodiment, there may be multiple levels or thresholds. For example there may be two levels or thresholds each indicating a different degree of danger or indicating a different degree of certainty that an abnormal event has occurred. In some cases, there may be a threshold for even suspected conditions may be recorded. In one embodiment, one threshold or level may indicate that there is a problem that is observed, which may record the event or condition without actually activating an alert and at a second level or threshold an alert may be activated. Activating the alert may include sending a communication indicating the alert. In an embodiment, alerts are sent for only conditions or abnormal motions that pass certain conditions or levels. In an embodiment, there may be a button to indicate that there is an abnormal motion occurring now, which may include an ask-for-help button. In an embodiment, the patient may be able to manually trigger the alert to be activated. In an embodiment, if the system detects a problem, but the patient is fine, the patient can press a button and indicate to ignore the alarm and that the call this false alarm. These thresholds can be adjusted by the patient/user. Each user may have different threshold or personal requirement on when to record and when to alert. The system may provide two adjustable sets of thresholds that can be modified. One set of thresholds is for sending an alert and one set of thresholds is for recording events that have seizure-like characteristics, but are expected not to be a seizure or for events that are near the borderline between being a seizure and not being a seizure. The input parameters may be entered manually by the user, stored, and reused, so that the user not need to input the parameters every time system is turned on or put in use.

In this approach, we utilize the input signal as is. The data can be windowed (overlapping) over short durations of time and the range of values examined. It is expected that non-abnormal-like activities exhibit value fluctuations only within a short range of values of acceleration (or gyro sensor), that is, the difference between the minimum acceleration value and the maximum acceleration value over a short period of time is bounded by two relatively close values. On the other hand, seizure-like activities exhibit a larger fluctuation and the difference between the minimum acceleration value and the maximum acceleration value.

There at least are four strategies to implement the detection system. Any one of these four strategies is sufficient and can be used to do the detection (1) learning based and/or (2) rules, conditions and/or logic based, (3) probabilistic/statistical models and detection methods and (4) analysis of local, regional, global features which include both data and temporal information. A hybrid of any combination these four strategies can also be used. Abnormal motion detection system may be based on learning system and incorporate supervised or unsupervised learning, which may include one or more neural-networks, machines that perform pattern recognition methods and/or support vector machines, for example. In embodiments including unsupervised learning, positive and negative data samples are provided to abnormal motion detection system as training examples for classifying patterns of behavior as seizure or non-seizure motion patterns. After being fed the training examples, abnormal detections system is able to make a determination as to whether other motion patterns are associated with unusual motions. By using unsupervised learning, after a training session and/or after learning from experience with actual motion patterns of user/wearer, abnormal motion detection system is able to detect abnormal having motion patterns that do not have features that are otherwise common amongst most other abnormal-like activities. For example, a neural-network or a Support Vector Machine can be trained based on positive and negative data samples.

Alternatively, the detection system or algorithm can be based on rules, conditions, or equations and pre-defined logic that is user independent. For example, the rule or logic can be to compute the local peaks of motion jerks or movements. If there are N jerks/movement happen within M seconds, then the motion may be determined to be seizure. For example, if # jerks are >5 within 3 seconds, then it is a seizure. Typical rules/logic will use one or more conditions or criteria based on: frequency/number of motion/jerks within a time frame, amplitude of the motion, continued change or duration of this motion, 1^{st} or 2^{nd} degree change of these values above. These rules/logic/conditions will change between the type of seizures like TonicClonic, Partial or Complex Seizures etc. Users can also adjust these rules/conditions/thresholds, to better suit their individual needs and type of seizures. There can also be defined set of types of conditions or detection rules templates, built in and users can select and chose and test different ones and pick the ones they like most.

In an embodiment, the motion data form cameras and/or from motion detector is transformed into the frequency domain, via a Fourier transform, or wavelet transform. In the frequency domain, the motion data is analyzed to see whether the motion data includes unique features in the frequency domain (such as larger coefficients corresponding to high frequency components) that are expected to be found in motion data from an abnormal motion. Performing a Fourier transform on a set of data taken within a particular window of time may be taken, (which may be referred to as a “widowed Fourier transform” and) which may capture the local nature of the signal. Similarly, orthogonal wavelet transforms (such as Daubechies) or another transform of the motion data may be taken (which may also provide a local representation of the signal in terms of the set of basis functions). The transformed may then be scanned for large coefficients of basis vectors that are expected to be associated with a seizure. The recognition seizure motion patterns in the frequency domain may be performed based on a supervised or unsupervised learning.

In an embodiment there may be a bank of motion detectors, which may include any combination of cameras or motion detector, other hybrid motion detectors, and/or other motion detectors, as described above, each running in parallel. The usage of a bank of motion detectors creates a system that is very robust and that detects seizures with a high level of accuracy.

In an embodiment, an accelerometer/gyro in a watch measures the accelerations in the three directions, aₓ, a_{y}, and a_{z}. Optionally, from the individual components that magnitude can be computed from a=SQRT(aₓ²+a_{y}²+a_{z}²). Optionally, the magnitude may be used to compute the absolute first derivative of the acceleration v=|aₙ−aₙ₋₁|, which is equivalent to “jerk.” Or abnormal motion. Optionally, the absolute second derivative may be computed v′=|aₙ−2aₙ₋₁+aₙ₋₂|. The first and/or second derivative of each component may be computed and/or the first and/or second derivative of magnitude may be computed. In an embodiment, a count is performed of all of the peaks of the first and/or second derivative that occur during a specified time window that are above a certain threshold. If the number of peaks is greater than a threshold number, it is assumed that a seizure is occurring.

If a preset number of peaks in the amplitude of the acceleration are obtained within a certain time interval, then it is considered to be an abnormal motion. If less than the preset numbers of peaks in the amplitude of the acceleration are obtained within the time interval, then it is assumed that an abnormal motion did not occur. In an embodiment, the variation of each component of acceleration is analyzed separately (in addition to or instead of analyzing the magnitude of the acceleration). During an abnormal motion, in each component of the acceleration, the peaks may be more frequent and shorter than for the amplitude, and consequently, for each component of acceleration, the threshold for the number of peaks (that is considered indicative of an abnormal motion) in a given time period may be set higher and the threshold for the amplitude of the peaks (that is considered indicative of a seizure) may be set lower than for the magnitude. The positive and negative examples of motion patterns that are fed to a neural network or other learning algorithm may include each component of acceleration and/or the magnitude of the acceleration.

An accelerometer sensor provides the acceleration and orientation of the body, while a gyro, will capture the angle of rotations. The accelerometer is secured to the patient's hand, arm, legs, and/or any other part of body that shakes and has the seizure movements. The following steps are used to detect the seizure from the accelerometer data.

In step, the sensor data is obtained by a time based sampling of the motion. For example, 25 or 100 or 1,000 samples per second are collected. In an embodiment, the samples may be an amount of deflection of a cantilever. The data sampling may be in any one or all X, Y, and Z axes. The data per axis may be one dimensional numerical accelerometer data. The motion data may be sampled over time. Hence for the X and Y axes, there will be two data streams. For triple axes data from axes X, Y, and Z, there will be 3 data streams. In an embodiment, the amplitude and frequency are measured as part of the time based data stream. The amplitude, frequency, change of position, and acceleration may be measured from the accelerometer or other motion sensor data. The data may be tracked over time. The data can be tracked in every time interval or specified time internals may be skipped. The time intervals at which the data is sampled define the sampling frequency.

In optional, the path of the data is analyzed or plotted. Data such as points, position, acceleration, velocity, and/or speed are extracted. If the points, positions, velocities, and acceleration were already determined as part of step may be skipped.

In this step, an oscillation analysis is performed, as described above, may be used to determine whether an abnormal motion takes place. The frequency, amplitude, time, and/or path of the oscillation may be determined. Frequency may determine how frequently the sensor and objects oscillate. The amplitude may indicate the amount of distance that the objects move. The path may indicate the exact nature and movement patterns of the objects. The frequency, amplitude, and/or path may be used to analyze oscillations and decide if the movement is a seizure.

If it is determined that no seizure occurs, this method terminates. After termination method may restart on another set of data. In an embodiment, many instances of method may be performed concurrently on different set of data. For example, after a first instance of method starts working on one pair of images, a second instance may start working on the next set of data, which may come from the next available pair of images, before the first instance of method terminates. In an embodiment, method is repeated for each set of data until motion detector and/or the processor are turned off.

The detection algorithm can use any of one or more of the following mathematical methods. In one embodiment, the peak and amplitude of the oscillation are checked, and compared to thresholds value of the peaks and amplitude. In an embodiment, if the peak and/or amplitude are greater than the threshold, then a determination is made that the oscillation is associated with an abnormal motion. An absolute and/or relative threshold may be used to find abnormalities, which may indicate an abnormal motion.

In another embodiment, a search is made for repeated peaks and valleys in the one dimensional sensor data (for X, Y, and/or Z). This technology uses the motion vector patterns based algorithm. Repeated and distinguished peaks and valleys may indicate abnormal motion.

In another embodiment, a search is made for duplicate peaks on other axes. In other words, one axis may have stronger peaks while the others may have weaker peaks. In some cases all axes can be stronger or weaker. However, neighboring axes can provide a valuable confirmation when the peaks on one axis have corresponding peaks on another axis.

In another embodiment, software and/or hardware neural networks or other learning methods may determine abnormal patterns to detect abnormal motion. In another embodiment, exact template patterns or signal patterns can be used to match other signal patterns. A prior known abnormal pattern can be used to compare the signal pattern with the known template and if the pattern detected matches the known abnormal motion pattern within a given tolerance, then it is expected that an abnormal motion occurred. These neural networks or other machines using other learning methods, can be either supervised or unsupervised learning.

In another embodiment, the position may be analyzed by determining the first derivative and the second derivative of a signal that is indicative of the position as a function of time. The first and second derivative of the position signal may be monitored to determine whether the first and second derivative are within a range that is considered to be an average and/or normal change of position (an average and normal first derivative dx/dt and an average and normal rate of change of position, which is the 2^{nd} derivative, d²x/dt²). When the first and/or second derivatives are abnormal, or beyond a threshold, then an indication is generated that an abnormal event may have been detected. Additionally, if the periodic changes of the first and second derivative are outside of a certain range, it may be an indication that an abnormal motion has occurred. Periodic changes in the second derivative are the third derivative, which are the impulses, which may be used to characterize jerky/unusual motion. Similarly, if the third derivative (or another derivative) is beyond a threshold or is not within a range that is considered normal, an indication that a seizure occurred may be generated. Additionally, if the pattern of times at which the third derivatives rise above a certain threshold matches that of a known pattern for a seizure and/or occur at a frequency that is expected to be indicative of an abnormal motion, an indication that an abnormal has occurred in generated.

In another embodiment, statistical learning or probabilistic methods are used. Machine learning strategies based on Bayesian Network (Bayes net) and HMM (Hidden Markov Models) and other statistical learning or probabilistic methods can be used for detection of abnormal motion.

In another embodiment, local, regional, and/or global features are detected. The features may be a collection of data taken from a neighborhood of a signal with temporal information. Local features are characteristics of signal and/or data in a small region of the data. Local features are a function of time (e.g., the features of a plot of the signal as a function of time), regional features covers more time, and global features are an average or a collection of local/regional features over longer time. Both local and global features can be a combination of both shape and time/temporal based. The local and global features are detected and compared to known local, regional and global features that are expected to characterize abnormal motion features to determine whether an abnormal motion has occurred. If the local, regional and global features that are associated with abnormal motion occur, then a signal is generated indicating that unusual movement has occurred.

### Individual Person's Abnormal Motion Signature

In one mode or embodiment, a person's abnormal motion data or motion signature may be measured as an abnormal motion occurs. “Abnormal motion detection” parameters (frequency, amplitude, patterns) can be customized as an “Abnormal motion signature” for each user. This signature can be adjusted and configured for each user if required for higher accuracy, as opposed to standard factory defaults.

Instead of a fixed abnormal motion signature, the person's signature may be determined over time. Then the detection algorithm will adapt and fine-tune the abnormal motion detection parameters based on the individuals' signature patterns—such as frequency, amplitude, time, area, path, and/or other parameters).

Each of the three components of acceleration labeled X, Y, and Z, are measured in a reference frame that is stationary with respect to the wrist or body. For example a Graph shows 9 peaks with about 5 second. The threshold for the number of peaks within a window of 6 seconds should be 9 peaks or less. The magnitude for the peaks of the y component of acceleration is 6 cm/sec², and the threshold for a single component of acceleration should be less than 6 cm/sec². Peaks that were below a predetermined threshold were removed. If the numbers of peaks within a specified window of time are greater than predetermined number, it is an indication of abnormal motion.

The units for acceleration are m/sec² and the units for impulse or jerk/movements are m/sec³. In an embodiment, for at least some patients the threshold for the magnitude of acceleration may be about 7 m/sec². The threshold for a “jerk” or impulse, may be about a number less than 4 m/sec³ e.g., 3 m/sec³, 3.5 m/sec³, or 3.8 m/sec³. The threshold for the frequency of peaks in acceleration may 3/(3 seconds) (e.g., 1 Hz). The number peaks in a window of 3 seconds there should be at least 3 peaks. The threshold for the number of peaks in the impulse should be at least 3, and within a window of about 3 seconds there should be at least 3 peaks in the impulse/jerk. In other embodiments, the units and the values for the thresholds above may be proportional to those given above.

### Reminders, Notifications, Alerts to Body Worn Devices

In some embodiments, the invention may be used for sending reminders and notifications from the outside to the body worn devices. The body worn devices are the same devices mentioned in the section 3. It can be one of these body worn devices as pendant, arm band device, watch, on the hat and so on (see FIG. 1).

### Technical Details and Architecture & Components

This embodiment has 3 types of architecture based on the number of components. Here the main components are:

1: The device (body worn)
2: The mobile phone
3: Cloud/Internet server

### Architecture Type 1: Device Only

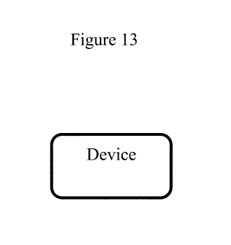

In this case the body worn device decides to send notification by itself from inside and it sends it to the device user. FIG. 13 shows an example of a device only architecture.

### Architecture Type 2: Device+Mobile Phone

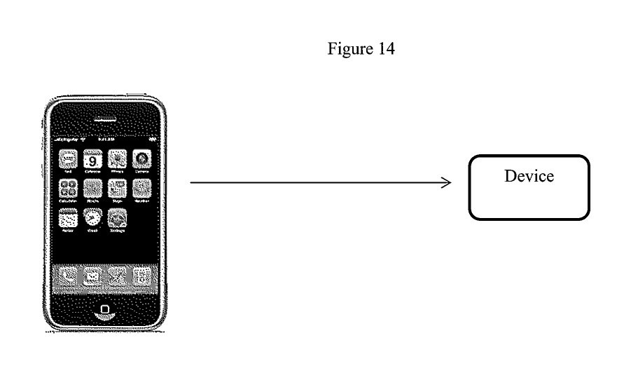

In this case, the mobile phone has some program running and it decides to send the notification to the body worn device. FIG. 14 gives an example of a device+mobile phone architecture.

### Architecture Type 3: Device+Mobile+Cloud/Internet Server (or any External System)

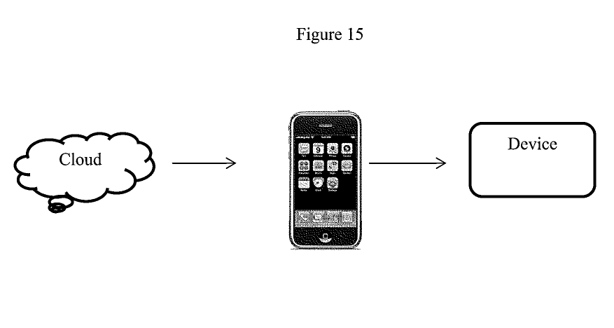

In this case, the Cloud server or any PC server on the net, decides to send a notification to the body worn device. It sends to the mobile phone. The mobile phone reflects and relays it to the body device. The cloud can be any external system or PC sending notification directly or via the cloud service to the device. FIG. 15 gives an example of a device+mobile+cloud/internet server architecture.

### Architecture Type 4: Cloud (or any External System)+Device

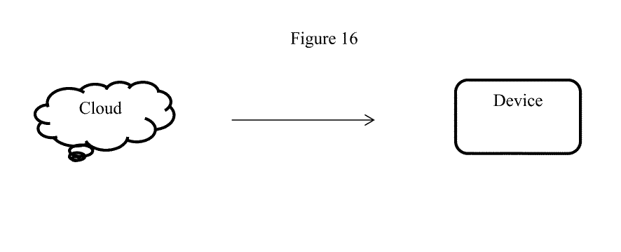

The device as the 3G/2G/4G modem and can directly access and talk to device. In this case the cloud server will directly send the notification to the device. Again, the cloud can be any external system or PC sending notification directly or via the cloud service to the device. FIG. 16 gives an example of a cloud (or any external system)+device architecture.

There can be an in-between device in some cases. This in-between device or messenger can be a mobile phone or PC or another compute or communication device.

### Communication Types/Medium

In this embodiment, the device typically has one of the 3 Wireless connection possibilities:
BlueTooth
Wifi
2G/3G/4G/5G/Cell modem to the phone network

For each case, the way we connect and send messages are different as mentioned below.

### Reminder Origination

The reminder origination or initial decision can be done in any one of the 4 places:
Inside from the body worn device. The Device by itself has a decision to send a notification. This can be based on the time or decision or reminder or some event based notification
From the mobile phone. The phone has some software running, which can an application, or background process or operating system etc. It decides to send a message and to be notified on the body device.
On the Cloud server. The cloud server on the internet has the decision or method—to send a notification or message
Outside world. This can be any Internet device, or Mobile phone or PC or system—which wants to send a notification

### Ways to Alert or Inform

This can be done by one of the following means:

1: There is a buzzer with an audible notification or speaker
2: It can be a vibrator to shake and inform the user of the notification
3: A display on the visual display screen
4: A light or visible blinking or light informing this notification. The light can be color coded into a red/green/blue, and so on.

### Type of Reminders and Notification:

1: A medication reminder for an action at a certain time
2: To make a call to someone
3: To execute and do a task
4: or simply it is on the hour

It can be any piece of reminder or notification or message to be sent to the body device.

### Notification/Communication Package

The notification is sent via a package of data from the mobile or cloud server to the device. The device via Bluetooth or Wi-Fi or 3G/4G/any-mobile communication medium, receives it. The typical package of data will have this following information:
Some time and package size info
Header or command
Data or information to execute the command
Acknowledge info

Some are optional and not fully required.

Notification Execution

Upon receiving the notification, the device will execute the notification to the user. This notification can be as mentioned earlier, can be any one or more of these:
Buzzer or beep
Speaker
Vibration
Lights on/blinking
Display
Etc.

The notification is made on the body device using any of these form selected.

### A Complete Working Instruction for a Notification Reminder

Let's talk thru how exactly one of the notification reminder will work on a body device.

For example the body device can be a pendant or a watch.

The notification reminder can be a “medication reminder on time”.

### Type 2: Mobile Phone->Device

1. In this case the mobile phone has the program running and decides at a specific time that it is time for medication.
2. It creates the message packet in that format and creates a notification.
3. Then it pushes the notification via Bluetooth or Wifi.
4. The device in this case can be a pendant or watch, which will continuously listen to the communication port for these commands or notification events.
5. Then the notification communication arrives,
6. Then the data packet is parsed and understood
7. Then the notification is executed and delivered to the user in the one of the following form: Buzzer with a beep; and/or via a vibration on the hand or belt; and/or a small message on the screen to inform of the notification.

### Type 3: Cloud->Mobile Phone->Device

1. In this case cloud server decides that it is time to send a notification.
2. It creates the message packet in that format and creates a notification.
3. It then sends a notification to the mobile phone
4. The mobile phone has the program running and listens to the cloud server and relays the message.
5. Then it pushes the notification via Bluetooth or Wifi.
6. The device in this case can be a pendant or watch, which will continuously listen to the communication port for these commands or notification events.
7. Then the notification communication arrives,
8. Then the data packet is parsed and understood
9. Then the notification is executed and delivered to the user in the one of the following form: Buzzer with a beep; and/or via a vibration on the hand or belt; and/or a small message on the screen to inform of the notification.

Alternatively the cloud server can directly talk to the body device.

The following activities are happening in these devices

### In Body Device

The device is constantly running or waking up every N seconds and listens to the communication port. It then parses and executed the notification and informs the user.

### In Mobile

In the mobile device, it can either decide to send notification (or) simply listen to the cloud server for notification messages and then it relays it to the body device.

### In Cloud

It can either decide to send notification (or) simply listen to the external PC or other phones or servers for notification messages and then it relays it to the body device (directly or via the mobile phone)

### Reminder/Notification Software and Program

The medication or other reminder program can be software that runs and has a list of possible notification and reminders set for specific time period or interval. It checks the current time or clock and decides that it is time to send that notification or reminder or medication requirement (or whatever) and sends that reminder or notification. This program can be run in any of the 3 places:
Inside the body device
In the mobile phone
In the cloud

### Scenarios and Typical Notification:

The following notification or use cases or scenarios will be included as part of this system. Much more combinations and variations are possible. This is just a short list of potential notification and not the only list:

1. Medication reminder
2. Body device is disconnected and needs attention
3. Time to take action and respond to a query
4. The last medical reason needs attention, perhaps blood pressure or weight
5. Call someone
6. An abnormal motion or event has occurred and needs to be sent
7. An abnormal event has occurred and want to confirm or cancel
8. Some family member wants to talk or connect
9. Emergency SOS will be sent out. Are you OK?
10. Unusual activity and needs confirmation or acceptance or support
And many more such notifications

### Several Embodiments and Forms

The whole invention can be applied to all 3-4 forms of applications or utilities or solutions:
1. A whole system. An independent and self-functioning system (including hardware, firmware, sensors, software and algorithms)
2. A method or methodology, for making technical product and making a viable solution or component
3. Algorithms—primarily the software, math processing and getting detection, recognition and alerting to work and the method to do the software
4. A module with one of more of these method or algorithm or a system component. (including any/some hardware, firmware, sensors, software and algorithms)

Embodiments of these above forms are viable and possible and presented in this invention. A utility patent can be followed or filed based on any of the above embodiments or forms.

### Check & Notify the Body Device Location

This method describes how to check and notify the body worn device location. This device can be any one of the devices as in Section 3 and FIG. 1.

The device may or may not have a GPS information or chip.

Hence there are 2 cases:

1: Device has the GPS chip
2: Device does not and mobile phone does

These devices will have regular heart beat and messaging protocol to send the “alive” and “OK” and current status message to the phone or cloud servers. A GPS location can also be sent.

Or, it can be done on demand basis, i.e. when the user or external system, asks for

1: Is it an active user or
2: Where is the body device (or phone or user)

There are 2 ways to execute this:

### 1: On Demand Delivery

In the next regular command time or messaging, the server will inform the body device (or mobile phone) to send the GPS location in the next message

Then in the next message, the GPS location information is sent to the caller

### 2: Continuous Location Streaming

In this case the location information is turned on, and hence the body device (or Phone) will continuously stream the GPS location to the caller.

Either case the caller can get the device location and device activity, is the device moving or not.

Device moving or not, can be determined by the motion of the device or by the GPS location movement from the location information.

This location information can be displayed on the caller device or phone or web. Or it can be sent via an SMS message as the answer to the previous question or request for information.

Alternatively, this can also be by permission basis. When the external user is asked for location information, the body worn device can pop the message for notification confirmation and get permission. User can accept or decline. Then the location information is forwarded or declined.

These are the methods to send the location notification or answers or confirmation via the body worn devices and external world.

1. An intelligent device, configured to be worn on the body of a human user, comprising at least one processor, at least one an output mechanism, and at least one sensor, wherein said processor is configured to use data from said at least one sensor to analyze said user's actions;
and wherein said processor is further configured to detect abnormal body motion and events.

2. The device of claim 1, wherein said device further comprises a mobile phone configured to perform motion analytics.

3. The device of claim 1, wherein said device further comprises a remote server configured to perform motion analytics.

4. The device of claim 1, wherein said device is further configured to output alerts and notifications when said abnormal body motion or events are detected.

5. The device of claim 1, wherein said abnormal body motion or events comprise at least one disorder selected from the group consisting of falling, fainting, sudden movement, accidents, Parkinson's disease, tremors, trembling, absence of movement, myoclonic jerks, and partial complex seizures.

6. The device of claim 1, wherein said device and output mechanism is further configured to either receive or transmit at least one of notification reminders, notifications, medication reminders, activity confirmations, health analysis, health checks, from either inside the device, from a mobile phone, or from an internet server.

7. The device of claim 1, wherein said processor is further configured to function as a medication reminder;
said device further configured to receive a medication reminder sent from outside or insider to the body worn device;
said device further configured with at least one medical time reminders;
wherein when the time reaches said at least one medical time reminder, then altering the user via said at least one output mechanism; or
wherein said device is further configured to receive at least one medication reminder from an outside device, and then alert the user using said at least one output mechanism.

8. The device of claim 7, wherein said device is a mobile phone.

9. The device of claim 1, wherein said device is further configured to receive an abnormal event status inquiry from an outside device, and wherein said device is configured to use its at least one processor to determine if said abnormal event occurred, and respond to said abnormal event status inquiry with the determination if said abnormal event occurred using said at least one output mechanism.

10. The device of claim 9, wherein said device is further configured to query the user using said at least one output mechanism to determine the recent history, medication status, medical status, or psychological history of said user.

11. The device of claim 9, further using either a mobile phone, Internet server, or other external computerized system to transmit an abnormal event status inquiry to said outside device, or receive said response to said abnormal event status inquiry.

12. A method of using a body worn intelligent device, worn by a human user, to determine the risk or propensity to develop at least one chronic condition selected from the group consisting of Parkinson's disease, depression, obesity, and diabetes, said method comprising:
obtaining an intelligent device, configured to be worn on the body of a human user, comprising at least one processor, at least one an output mechanism, and at least one sensor, wherein said processor is configured to use data from said at least one sensor to analyze said user's motions, thereby producing motion analysis data;
wherein said processor is further configured to detect abnormal body motion and events;
wherein said at least one sensor comprises at least one motion sensor;
further using the motion analytics results from said at least one motion sensor to determine the user's risk or propensity to develop at least one of said chronic conditions.

13. The method of claim 12, further analyzing said motion over a period of time to identify trends in said at least one chronic condition.

14. The method of claim 12, further analyzing said motion, thereby producing motion analysis data, and comparing said motion analysis data to a set of reference motion data for healthy individual individuals, and further detecting deviations from said set of reference motion data.

15. The method of claim 12, further comparing said motion analysis data to a chronic condition data set obtained from individuals with at least one of said chronic conditions, and determining similarities.
